SEQUENCE LISTING (47 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 4 - protein, 107 aa, Artificial Sequence
  Description of Artificial Sequence: Synthetic polypeptide
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, Artificial Sequence
  Description of Artificial Sequence: Synthetic peptide
RASKSINKYLA

SEQ ID NO: 6 - protein, 7 aa, Artificial Sequence
  Description of Artificial Sequence: Synthetic peptide
SGSTLQS

SEQ ID NO: 7 - protein, 9 aa, Artificial Sequence
  Description of Artificial Sequence: Synthetic peptide
QQHNEYPLT

SEQ ID NO: 8 - protein, 121 aa, Artificial Sequence
  Description of Artificial Sequence: Synthetic polypeptide
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, Artificial Sequence
  Description of Artificial Sequence: Synthetic peptide
GYHFTSYWMH

SEQ ID NO: 10 - protein, 17 aa, Artificial Sequence
  Description of Artificial Sequence: Synthetic peptide
VIHPNSGSINYNEKFES

SEQ ID NO: 11 - protein, 12 aa, Artificial Sequence
  Description of Artificial Sequence: Synthetic peptide
ERDSTEVLPMDY

SEQ ID NO: 12 - DNA, 321 bp, Artificial Sequence
  Description of Artificial Sequence: Synthetic polynucleotide
gatgtccagataacccagtctccatcttatcttgctgcatctcctggagaaaccattactattaattgcagggcaagtaagagcattaacaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacaacataatgaatacccgctcacgttcggtgctgggaccaagctggagctgaaa

SEQ ID NO: 13 - DNA, 363 bp, Artificial Sequence
  Description of Artificial Sequence: Synthetic polynucleotide
caggtccaactgcagcagcctggggctgagctggtaaagcctggggcttcagtgaagttgtcctgcaagtcttctggctaccatttcaccagctactggatgcactgggtgaagcagaggcctggacaaggccttgagtggattggagtgattcatcctaatagtggtagtattaactacaatgagaagttcgagagcaaggccacactgactgtagacaaatcctccagcacagcctacatgcaactcagcagcctgacatctgaggactcggcggtctattattgtgcaggagagagagattctacggaggttctccctatggactactggggtcaaggaacctcagtcaccgtctcctca

SEQ ID NO: 14-30 - skipped (17 entries)

SEQ ID NO: 31 - protein, 121 aa, Artificial Sequence [sequence omitted]

SEQ ID NO: 32 - protein, 121 aa, Artificial Sequence [sequence omitted]

SEQ ID NO: 33 - protein, 121 aa, Artificial Sequence
  Description of Artificial Sequence: Synthetic polypeptide
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 34 - protein, 121 aa, Artificial Sequence [sequence omitted]

SEQ ID NO: 35 - protein, 107 aa, Artificial Sequence [sequence omitted]

SEQ ID NO: 36 - protein, 107 aa, Artificial Sequence [sequence omitted]

SEQ ID NO: 37 - protein, 107 aa, Artificial Sequence
  Description of Artificial Sequence: Synthetic polypeptide
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, Artificial Sequence [sequence omitted]

SEQ ID NO: 39 - protein, 229 aa, Artificial Sequence
  Description of Artificial Sequence: Synthetic polypeptide
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, Artificial Sequence
  Description of Artificial Sequence: Synthetic polypeptide
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41-46 - skipped (6 entries)

SEQ ID NO: 47 - protein, 327 aa, Artificial Sequence
  Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2024/0059765 A1
(43) Pub. Date: Feb. 22, 2024

(54) COMPOSITIONS AND METHODS FOR TREATING OCULAR DISEASES

(71) Applicant: Annexon, Inc., Brisbane, CA (US)

(72) Inventors: Ted Yednock, Brisbane, CA (US); Anita Grover, Brisbane, CA (US); Lori Taylor, Brisbane, CA (US)

(21) Appl. No.: 18/265,380

(22) PCT Filed: Dec. 3, 2021
(86) PCT No.: PCT/US21/61755

§ 371 Date: Jun. 5, 2023

Related U.S. Application Data
(60) Provisional application No. 63/121,629, filed on Dec. 4, 2020.

(51) Int. Cl. C07K 16/18; A61P 27/06

(52) U.S. Cl. CPC C07K 16/18; A61P 27/06; C07K 2317/56; A61K 2039/54

(57) ABSTRACT

The present disclosure relates generally to compositions and methods of preventing, reducing risk of developing, or treating an ocular disease (e.g., glaucoma or age-related macular degeneration). The age-related macular degeneration may be geographic atrophy.

### RELATED APPLICATIONS

This patent application claims priority to U.S. Provisional Patent Application No. 63/121,629, filed Dec. 4, 2020, which is hereby incorporated by reference in its entirety.

### BACKGROUND

Glaucoma and Age-related macular degeneration (AMD) are the leading cause of irreversible blindness globally. Geographic atrophy (GA), also known as atrophic AMD or advanced dry AMD, is an advanced form of AMD. The global prevalence of glaucoma for the population aged 40-80 years is 3.54% (95% CrI, 2.09-5.82). By 2040, the prevalence is projected to rise to 112 million worldwide and 4.7 million in North America. Currently approved glaucoma treatments are limited to lowering intraocular pressure (IOP). Surgery, laser treatment or IOP lowering agents are all commonly used. However, even with good control of IOP with medication or surgery, many glaucoma patients continue to experience progressive visual loss. Further, there are no approved treatments or therapies for GA. Therefore, there is a significant unmet need for neuroprotective treatments for patients in whom control of IOP is insufficient to prevent the loss of ganglion cells, axons or synapses that lead to progressive and irreversible vision loss. Thus, there is a need for new therapies to prevent, reduce the risk of developing, and treat ocular diseases (such as glaucoma and AMD, including geographic atrophy).

### SUMMARY

The present disclosure is generally directed to compositions and methods of preventing, reducing risk of developing, or treating an ocular disease (e.g., glaucoma or age-related macular degeneration, such as AMD, including geographic atrophy) in a human patient. Such methods include administering to the patient a composition comprising about 1 mg to about 10 mg of an anti-C1q antibody via an intravitreal injection, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7; and a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the heavy chain variable domain comprising an amino acid sequence selected from SEQ ID NO: 8 and 31-34. In some embodiments, the antibody comprises a light chain variable domain comprising an amino acid sequence with at least about 95% homology to the amino acid sequence selected from SEQ ID NO: 4 and 35-38, and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, and a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence selected from SEQ ID NO: 4 and 35-38, and a heavy chain variable domain comprising an amino acid sequence selected from SEQ ID NO: 8 and 31-34. The antibody may be a monoclonal antibody, a humanized antibody, a human antibody, a chimeric antibody, an antibody fragment, or antibody derivative thereof. The antibody fragment may be a Fab fragment, a Fab′ fragment, a F(ab′)2 fragment, a Fv fragment, a diabody, or a single chain antibody molecule. In some embodiments, the Fab fragment comprises a heavy chain Fab fragment of SEQ ID NO: 39 and a light chain Fab fragment of SEQ ID NO: 40.

In some embodiments, the antibody is administered once a week, once every other week, once every three weeks, once a month, once every 4 weeks, once every 6 weeks, once every 8 weeks, once every other month, once every 10 weeks, once every 12 weeks, once every three months, or once every 4 months. In some embodiments, the antibody is administered for at least 6 months, at least 7 months, at least 8 months, at least 9 months, at least 10 months, at least 11 months, or at least 12 months.

In some embodiments, the administered composition comprises about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of the anti-C1q antibody. The administered composition may comprise about 1 mg to about 5 mg of the anti-C1q antibody. The administered composition may comprise about 1 mg to about 2.5 mg, about 2.5 mg to about 5 mg, about 5 mg to about 7.5 mg, or about 7.5 mg to about 10 mg of the anti-C1q antibody. The administrated composition may comprise about 5 mg of the anti-C1q antibody. The administrated composition may comprise about 10 mg of the anti-C1q antibody.

In some embodiments, the ocular disease is glaucoma or age-related macular degeneration, such as geographic atrophy.

### DESCRIPTION OF THE FIGURES

FIG. 1 shows human C1q binding assay. Binding of Mab2-Fab, FabA and Mab2 to human C1q in a one-sided ELISA. Bound antibody or Fab molecules were detected using an enzyme-tagged anti-human-Fc or anti-human kappa antibody followed by enzyme substrate. The antibodies showed comparable binding affinity for human C1q. EC50 for Mab2-Fab, FabA and Mab2 =4.4, 2.5, and 4.9 ng/mL, respectively (range of 34-95 pM).

FIG. 2 shows that FabA inhibits the classical, but not the lectin and alternate complement pathways. FabA and Mab2 were evaluated for their ability to inhibit the classical, lectin and alternate pathways using ELISA based assay kits from Eurodiagnostica (Weislab™). The wells are coated with specific activators of the classical pathway (IgM), the lectin pathway (mannan), or the alternate pathway (lipopolysaccharide), and activation of all pathways was assessed using a C5b-9 terminal complex detection antibody. An inhibitory antibody against C5 was used as a positive control. FabA and Mab2 selectively block the classical pathway with an IC50 of ≤0.3 μg/mL, while anti-C5 inhibits all three pathways. FIG. 3 shows inhibition of hemolysis of IgM-coated RBC in human serum.

Sheep RBCs, pre-sensitized with a surface-reactive polyclonal IgM antibody, were co-incubated with human serum (diluted 100×) at 37° C. for 20-30 minutes. RBC hemolysis was quantified by measuring release of hemoglobin, and is expressed as a percentage of hemolysis induced by no-treatment.

FIG. 4 shows reduction in the number of damaged axons in the optic nerves of eyes treated with Mab1-Fab, Mab1, or Mab2. Increased IOP was induced in one eye of each animal by injection of 1 μl of 6 μm polystyrene beads, 1 μl of 10 μm polystyrene beads (Polybead Microspheres; Polysciences, Inc., Warrington, PA, USA) and 1 μl of viscoelastic solution (10 mg/mL sodium hyaluronate; Advanced Medical Optics Inc., USA) into the anterior chamber of the eye on Day 1. The contralateral eye was left untouched to serve as a control. Antibodies Mab2, Mab 1, and Mab1-Fab (Fab derived by enzymatic digest of Mab1) vs. saline were administered to the microbead-injected eyes intravitreally, one day prior to microbead injection and one week later (Day 0 and Day 7; 2 μL of a 10 mg/mL antibody saline solution for each injection vs. saline alone). Two weeks post injury, optic nerves were collected from animals (perfused with saline and 4% paraformaldehyde), postfixed with 4% paraformaldehyde and 1% osmium, dehydrated in ascending alcohol concentration and placed in 1% uranyl acetate/ethanol. Nerves were embedded in epoxy resin and semi-thin sections (1 um) were cut. The total number of degenerating axons was estimated using StereoInvestigator software (MicroBrightfield, Inc, VT, USA). Scale bar=20 um. Mab1-Fab and Mab2 both significantly reduced formation of damaged axons in the optic nerve, while antibody Mab 1 showed a similar trend.

FIGS. 5A-5D show protection of photoreceptor neuron loss and retinal function in a mouse photodamage model with Mab1 antibody. FIG. 5A shows photodamage model in mouse for 7 days followed by intravitreal (IVT) administration of Mab1 antibody and assessment of retinal function and histology at Day 14. Mice were administered 1 μL of 7.5 mg/mL Mab 1 or isotype control antibody via IVT administration on Day 7. FIG. 5B shows that Mab1 treatment led to a significant reduction in Tunel+ve photoreceptor cells in the outer nuclear layer of the retina, when compared to isotype control. FIG. 5C shows that Mab1 treatment led to an increase in the number of photoreceptor cell rows in the outer nuclear layer, when compared to isotype control. FIG. 5D shows that Mab1 antibody treatment led to a significant increase in the A-wave and B-wave in electroretinogram on Day 14, when compared to isotype control antibody.

FIG. 6 shows free C1q in aqueous humor after a single IVT injection.

### DETAILED DESCRIPTION

### General

The present disclosure is generally directed to compositions and methods of preventing, reducing risk of developing, or treating an ocular disease (e.g., glaucoma or age-related macular degeneration, such as AMD, including geographic atrophy).

Disclosed herein is a recombinant humanized Immunoglobulin G (IgG1) antigen-binding fragment (Fab) that inhibits the classical complement cascade, without affecting the lectin or alternative complement pathways. Anti-C1q Fab (e.g., FabA, an anti-C1q Fab comprising heavy chain Fab fragment of SEQ ID NO: 39 and light chain Fab fragment of SEQ ID NO: 40) is developed as an intravitreally (IVT) administered agent for the treatment for ophthalmologic diseases such as glaucoma and AMD, including geographic atrophy (GA). The hypervariable regions derived from the murine antibody MI (Mab1 antibody comprising heavy chain variable domain of SEQ ID NO: 3 and light chain variable domain of SEQ ID NO: 7) were expressed as a human IgG1 Fab fragment construct (FabA). A full-length human IgG4 antibody (Mab2, an antibody comprising heavy chain variable domain of SEQ ID NO: 8 and light chain variable domain of SEQ ID NO: 4) comprising the hypervariable regions derived from Mab1 was also expressed. Mab1 and Mab2, as well as their Fabs (Mab 1-Fab and Mab2-Fab), were used as surrogate molecules for FabA in pharmacology studies. As a monovalent Fab construct lacking Fc heavy chain constant domains 2 and 3 (CH2 and CH3), FabA cannot bind to C1q through Fc domain interactions. Furthermore, with only a single antigen-binding arm, FabA does not exhibit agonistic activity for C1q over a broad range of concentrations of FabA.

The complement cascade is a critical component of innate immunity and can be activated through 3 distinct pathways: the classical, lectin, and alternative complement pathways. All 3 pathways lead to the activation of complement component C3, which ultimately leads to immune cell recruitment, inflammation, membrane lysis through the membrane attack complex, and cell death.

C1q, the initiating molecule of the classical complement cascade, has been implicated in the initiation and propagation of neurodegenerative disease, including glaucoma and AMD, including GA. C1q inhibition may block initiation of the classical complement cascade and slow down neuronal and synaptic damage via directly reducing damage to nerve cell membranes and by reducing the inflammatory consequences of complement activation.

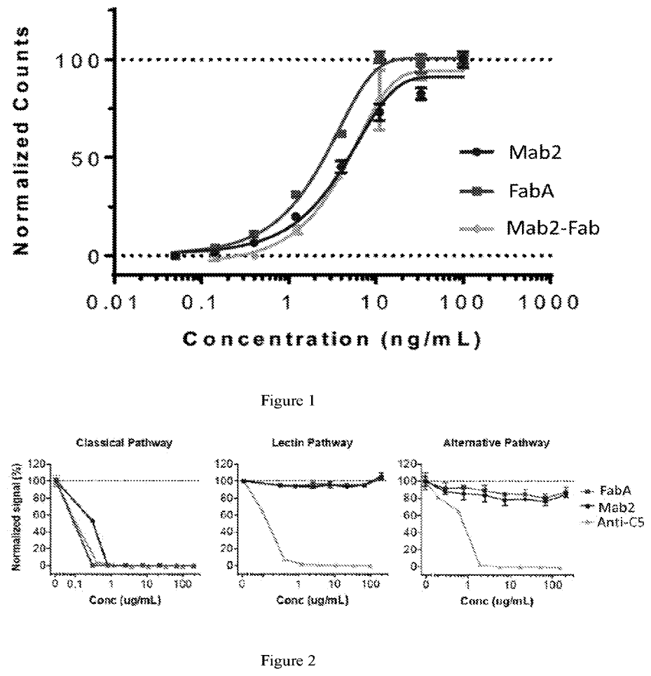

Mab2-Fab and/or FabA exhibit high affinity binding to human C1q as measured by Biacore (<10 pM) and by enzyme-linked immunosorbent assay (ELISA) (40-50 pM; FIG. 1). Mab1 binds to the isolated globular head domains of C1q, but not to C1q's collagen tail (as determined by ELISA). Consistent with this finding, Mab1 inhibits substrate interactions mediated by C1q's globular head domain (IgM, C-reactive protein [CRP], and phosphatidylserine); and FabA inhibits C1q functional interaction with immunoglobulin M (IgM)-coated red blood cells (RBCs) (blocking hemolysis; FIG. 3). Antibody Mab1 specifically recognizes C1q, showing no binding to the other complement components (C3b and C5), or to other C1q/tumor necrosis factor (TNF) superfamily members, including TNF and adiponectin, a protein that shares the highest sequence identity to C1q in its globular head domain. Consistent with these results, FabA does not inhibit the lectin complement pathway, which is initiated by the mannose-binding lectin (MBL, another member of the C1q/TNF superfamily), nor does it inhibit the alternative complement pathway (initiated by C3b) (FIG. 2).

Glaucoma includes a group of progressive eye disorders that damage the optic nerve, ultimately leading to loss of vision. In primary open-angle glaucoma, a primary contributor to optic nerve damage is elevated intraocular pressure (TOP) which, over time, leads to a characteristic loss in vision on visual field testing This vision loss is due to a progressive degeneration of: (1) retinal neurons, or ganglion cells, in the retinal ganglion cell (RGC) layer; (2) their axons, in the retinal nerve fiber layer (RNFL); and (3) a reduction in the number of neuronal synapses, predominantly in the inner plexiform layer of the retina.

Currently approved glaucoma treatments are limited to lowering intraocular pressure (IOP). Surgery, laser treatment or IOP lowering agents are all commonly used. However, even with good control of TOP with medication or surgery, many glaucoma patients continue to experience progressive visual loss. In the Collaborative Initial Glaucoma Study (CIGTS), glaucoma patients were randomized to initial treatment with either trabeculectomy (surgery) or topical medications. Despite a mean IOP ranging from 17.1 to 18.3 mmHg in the medication group or 13.8 to 14.4 mmHg in the surgery group over the course of the study, the proportion of patients who had a substantial worsening (3 dB or more in mean deviation by Humphrey visual field [HVF]) was 23.1% and 34.1%, respectively at 9 years. In the Early Manifest Glaucoma Trial, patients were randomized to argon laser trabeculoplasty combined with a topical beta-blocker versus no treatment until progression was observed. In the group randomized to initial treatment, 45% of patients had progressed at Year 6, based on HVF or optic nerve examination, despite a 25% decrease from baseline in IOP. These data establish a significant unmet need for 15 neuroprotective treatments for patients in whom control of IOP is insufficient to prevent the loss of ganglion cells, axons or synapses that lead to progressive and irreversible vision loss.

C1q recognizes certain pathogens, modifications of self-antigens, antigen-bound antibodies, or specific molecules on the surface of cells. In normal aging, C1q accumulates on synapses—perhaps those weakened by age or neuronal stress—and following various pathophysiological stimuli can trigger activation of the classical complement cascade, leading to the inappropriate elimination of synapses. This aberrant inflammatory response, associated with synapse removal, is termed complement-mediated neurodegeneration (CMND). CMND has been implicated in Alzheimer's disease, schizophrenia, Huntington's disease, frontotemporal dementia, spinal muscular atrophy and in glaucoma. With degenerative stress in the retina, C1q activation leads to synapse elimination and contributes to the loss of RGC's and the optic nerve. C1q elevation and complement activation have been observed in human glaucomatous retina, demonstrated by proteomic analysis and by histological staining. CMND has also been reported in rat, mouse, and dog models of glaucoma. In a chronic, spontaneous mouse model of glaucoma (DBA/2J mice), retinal C1q accumulation and synapse loss occur early in the disease process; genetic deletion of C1q is protective, significantly delaying loss of RGC and degeneration of the optic nerve. These results were replicated in another mouse model involving acute elevation of IOP with microbead injection into the anterior chamber of the eye; either genetic deletion of C1q or pharmacological inhibition with direct injection of a complement inhibitor into the eye, protected against neuronal cell loss and retinal degeneration. Another study examined retinal degeneration following transient ischemia induced by brief supra-elevation of IOP and again, RGC loss and retinal thinning were ameliorated in C1q knock out mice.

Age-related macular degeneration (AMD) is a leading cause of blindness globally. GA, also known as atrophic AMD or advanced dry AMD, is an advanced form of AMD resulting in the progressive and irreversible loss of central retinal photoreceptors, retinal pigment epithelium, and choriocapillaris, leading to vision loss. Currently, there are no approved treatments or therapies for GA.

Complement appears to be genetically linked to AMD with polymorphisms identified in 6 distinct proteins that can alter complement pathway activity. By inhibiting the interaction of the target-binding heads of C1q, as with FabA, the activity of the classical pathway can be fully inhibited, leaving the lectin and alternative complement pathways intact. The C1q/classical complement pathway mediates elimination of unwanted synapses in development. In adults, C1q accumulates on synapses with age and disease and can aberrantly trigger synapse elimination, neuroinflammation and degeneration. Inhibition of C1q is protective in numerous models of neurodegeneration.

C1q, the initiating molecule of the classical complement cascade, has also been implicated in the initiation and propagation of GA. C1q accumulates with age in two separate and important GA-associated disease processes—on photoreceptor neuron synapses in the outer plexiform layer and on drusen. Increasing size and area of drusen, which are extracellular accumulations comprised of lipids and proteins from degenerating photoreceptor cell outer segments, are associated with risk of developing AMD. It has been shown that both classical and alternative complement pathway activation is evident on photoreceptor outer segments and drives retinal atrophy in GA. Aberrant C1q/classical pathway activity is associated with loss of photoreceptors in a photodamage-induced mouse model of GA. Local C1q produced by retinal microglia/macrophages has been demonstrated as an instigator of inflammasome activation and inflammation. Retinal C1q inhibition with FabA in this model slowed retinal atrophy, reduced photoreceptor loss, increased retinal thickness, and preserved retinal function. FabA provides a novel approach for the treatment of complement-mediated retinal degenerations including GA.

All sequences mentioned in the present disclosure are incorporated by reference from U.S. patent application Ser. No. 14/933,517, U.S. patent application Ser. No. 14/890,811, U.S. Pat. Nos. 8,877,197, 9,708,394, U.S. patent application Ser. No. 15/360,549, U.S. Pat. Nos. 9,562,106, 10,450,382, 10,457,745, International Patent Application No. PCT/US2018/022462 each of which is hereby incorporated by reference for the antibodies and related compositions that it discloses.

Full-length antibodies may be prepared by the use of recombinant DNA engineering techniques. Such engineered versions include those created, for example, from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The DNA encoding the antibody may be prepared by deleting all but the desired portion of the DNA that encodes the full length antibody. DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode antibodies include cells, such as hybridomas, that express the full length antibody. For example, the antibody may be isolated from a host cell that expresses an expression vector that encodes the heavy and/or light chain of the antibody.

Antibody fragments, including but not limited to Fab fragments, and/or antibody derivatives may also be prepared by the use of recombinant DNA engineering techniques involving the manipulation and re-expression of DNA encoding antibody variable and constant regions. Standard molecular biology techniques may be used to modify, add or delete further amino acids or domains as desired. Any alterations to the variable or constant regions are still encompassed by the terms ‘variable’ and ‘constant’ regions as used herein. In some instances, PCR is used to generate an antibody fragment by introducing a stop codon immediately following the codon encoding the interchain cysteine of C_{H}1, such that translation of the C_{H}1 domain stops at the interchain cysteine. Methods for designing suitable PCR primers are well known in the art and the sequences of antibody C_{H}1 domains are readily available. In some embodiments, stop codons may be introduced using site-directed mutagenesis techniques.

An antibody of the present disclosure may be derived from any antibody isotype (“class”) including for example IgG, IgM, IgA, IgD and IgE and subclasses thereof, including for example IgG1, IgG2, IgG3 and IgG4. In certain preferred embodiments, the heavy and light chains of the antibody are from IgG. The heavy and/or light chains of the antibody may be from murine IgG or human IgG. In certain other preferred embodiments, the heavy and/or light chains of the antibody are from human IgG1. In still other preferred embodiments, the heavy and/or light chains of the antibody are from human IgG4.

An antibody of the present disclosure may bind to and inhibit a biological activity of C1q, C1r, or C1s. For example, (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to B-amyloid, or (10) C1q binding to calreticulin. In other embodiments, the biological activity of C1q is (1) activation of the classical complement activation pathway, (2) reduction in lysis and/or reduction in C3 deposition, (3) activation of antibody and complement dependent cytotoxicity, (4) CH50 hemolysis, (5) a reduction in red blood cell lysis, (6) a reduction in red blood cell phagocytosis, (7) a reduction in dendritic cell infiltration, (8) inhibition of complement-mediated red blood cell lysis, (9) a reduction in lymphocyte infiltration, (10) a reduction in macrophage infiltration, (11) a reduction in antibody deposition, (12) a reduction in neutrophil infiltration, (13) a reduction in platelet phagocytosis, (14) a reduction in platelet lysis, (15) an improvement in transplant graft survival, (16) a reduction in macrophage mediated phagocytosis, (17) a reduction in autoantibody mediated complement activation, (18) a reduction in red blood cell destruction due to transfusion reactions, (19) a reduction in red blood cell lysis due to alloantibodies, (20) a reduction in hemolysis due to transfusion reactions, (21) a reduction in alloantibody mediated platelet lysis, (22) an improvement in anemia, (23) a reduction in eosinophilia, (24) a reduction in C3 deposition on red blood cells (e.g., a reduction of deposition of C3b, iC3b, etc., on RBCs), (25) a reduction in C3 deposition on platelets (e.g., a reduction of deposition of C3b, iC3b, etc., on platelets), (26) reduction in anaphylatoxin production, (27) a reduction in autoantibody mediated blister formation, (28) a reduction in autoantibody induced erythematosus, (29) a reduction in red blood cell destruction due to transfusion reactions, (30) a reduction in platelet lysis due to transfusion reactions, (31) a reduction in mast cell activation, (32) a reduction in mast cell histamine release, (33) a reduction in vascular permeability, (34) a reduction in complement deposition on transplant graft endothelium, (35) B-cell antibody production, (36) dendritic cell maturation, (37) T-cell proliferation, (38) cytokine production, (39) microglia activation, (40) Arthus reaction, (41) a reduction of anaphylatoxin generation in transplant graft endothelium, or (42) activation of complement receptor 3 (CR3/C3) expressing cells.

In some embodiments, CH50 hemolysis comprises human, mouse, and/or rat CH50 hemolysis. In some embodiments, the antibody is capable of neutralizing from at least about 50%, to at least about 95% of CH50 hemolysis. In some embodiments, the antibody is capable of neutralizing 50%, 60%, 70%, 80, 90%, or 100% of CH50 hemolysis. The antibody may also be capable of neutralizing at least 50% of CH50 hemolysis at a dose of less than 150 ng/ml, less than 100 ng/ml, less than 50 ng/ml, or less than 20 ng/ml.

Other in vitro assays to measure complement activity include ELISA assays for the measurement of split products of complement components or complexes that form during complement activation. Complement activation via the classical pathway can be measured by following the levels of C4d and C4 in the serum. Activation of the alternative pathway can be measured in an ELISA by assessing the levels of Bb or C3bBbP complexes in circulation. An in vitro antibody-mediated complement activation assay may also be used to evaluate inhibition of C3a production.

An antibody of the present disclosure may be a monoclonal antibody, a polyclonal antibody, a recombinant antibody, a humanized antibody, a human antibody, a chimeric antibody, a multispecific antibody, an antibody fragment thereof, or a derivative thereof. In some embodiments, the antibody is humanized antibody.

The antibodies of the present disclosure may also be an antibody fragment, such as a Fab fragment, a Fab′ fragment, a F(ab′)₂ fragment, a Fv fragment, a diabody, or a single chain antibody molecule. In some embodiments, the antibody fragment is a Fab fragment.

In some embodiments, antibodies are human monoclonal antibodies which may be prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In some embodiments, antibodies are humanized and/or chimeric monoclonal antibodies, which can be raised by immunizing rodents (e.g., mice, rats, hamsters and guinea pigs) with either (1) the native complement component (e.g., C1q) derived from enzymatic digestion of a purified complement component from human plasma or serum, or (2) a recombinant complement component, or its derived fragment, expressed by either eukaryotic or prokaryotic systems. Other animals can be used for immunization, e.g., non-human primates, transgenic mice expressing human immunoglobulins, and severe combined immunodeficient (SCID) mice transplanted with human B-lymphocytes.

Polyclonal and monoclonal antibodies are naturally generated as immunoglobulin (Ig) molecules in the immune system's response to a pathogen. A dominating format with a concentration of 8 mg/ml in human serum, the ˜150-kDa IgG1 molecule is composed of two identical ˜50-kDa heavy chains and two identical ˜25-kDa light chains.

Hybridomas can be generated by conventional procedures by fusing B-lymphocytes from the immunized animals with myeloma cells. In addition, anti-C1q antibodies can be generated by screening recombinant single-chain Fv or Fab libraries from human B-lymphocytes in a phage-display system. The specificity of the MAbs to human C1q can be tested by enzyme linked immunosorbent assay (ELISA), Western immunoblotting, or other immunochemical techniques.

The inhibitory activity on complement activation of antibodies identified in the screening process can be assessed by hemolytic assays using either unsensitized rabbit or guinea pig RBCs for the alternative complement pathway, or sensitized chicken or sheep RBCs for the classical complement pathway. Those hybridomas that exhibit an inhibitory activity specific for the classical complement pathway are cloned by limiting dilution. The antibodies are purified for characterization for specificity to human C1q by the assays described above.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

As used herein, administration “conjointly” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit biological activity, and antibody derivatives.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The L chain from any vertebrate species can be assigned to one of two clearly distinct types, called kappa (“K”) and lambda (“AS”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ϵ”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W. B. Saunders Co., 2000).

“Full-length antibodies” are usually heterotetrameric glycoproteins of about 150,000 daltons, comprising two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” molecule or cell is a molecule or a cell that is identified and separated from at least one contaminant molecule or cell with which it is ordinarily associated in the environment in which it was produced. Preferably, the isolated molecule or cell is free of association with all components associated with the production environment. The isolated molecule or cell is in a form other than in the form or setting in which it is found in nature. Isolated molecules therefore are distinguished from molecules existing naturally in cells; isolated cells are distinguished from cells existing naturally in tissues, organs, or individuals. In some embodiments, the isolated molecule is an anti-C1q antibody of the present disclosure. In other embodiments, the isolated cell is a host cell or hybridoma cell producing anti-C1q antibody of the present disclosure.

An “isolated” antibody is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In certain preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. An isolated antibody includes the antibody in situ within recombinant T-cells since at least one component of the antibody's natural environment will not be present. Ordinarily, however, an isolated polypeptide or antibody will be prepared by a process including at least one purification step.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous since they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5):1073-1093 (2004); Fellouse, Proc. Nat'l Acad. Sci. USA 101 (34):12467-472 (2004); and Lee et al., J. Immunol. Methods 284 (1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” and “whole antibody” are used interchangeably to refer to an antibody in its substantially intact form, as opposed to an antibody fragment or antibody derivative. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” or “antigen-binding fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8 (10):1057-1062 (1995)). Additional examples of antibody fragments include antibody derivatives such as single-chain antibody molecules, monovalent antibodies and multispecific antibodies formed from antibody fragments

An “antibody derivative” is any construct that comprises the antigen-binding region of an antibody. Examples of antibody derivatives include single-chain antibody molecules, monovalent antibodies and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fe region herein will preferably possess at least about 80% homology with a native sequence Fc region and/or with an Fe region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγR1, FcγRII, and FcγRIII subclasses, including allelic variants and alternatively spliced forms of these receptors, FcγRII receptors include FcγRIIA (an “activating receptor”) and FcpγRIIB (an “inhibiting receptor”), which have similar amino acid sequences that differ primarily in the cytoplasmic domains thereof. Activating receptor FcpγRIIA contains an immunoreceptor tyrosine-based activation motif (“ITAM”) in its cytoplasmic domain. Inhibiting receptor FcpγRIIB contains an immunoreceptor tyrosine-based inhibition motif (“ITIM”) in its cytoplasmic domain. (See, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9 (2):6591-6604 (2001).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three FIVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain Preferably, the sFv polypeptide further comprises a polypeptide linker between the V_{H} and V_{L} domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fe), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, NJ, 2003)). Indeed, naturally occurring camelid antibodies consisting of a heavy chain only are functional and stable in the absence of light chain See, e.g., Hamers-Casterman et al., Nature 363:446-448 (1993) and Sheriff et al., Nature Struct Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 5 91:3809-3813 (1994); Schier et al. Gene 169:147-155 (1995); Yelton et al. J Immunol. 155:1994-2004 (1995); Jackson et al., J. Immunol. 154 (7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

The term “inhibitor” refers to a compound having the ability to inhibit a biological function of a target biomolecule, for example, an mRNA or a protein, whether by decreasing the activity or expression of the target biomolecule. An inhibitor may be an antibody, a small molecule, or a nucleic acid molecule. The term “antagonist” refers to a compound that binds to a receptor, and blocks or dampens the receptor's biological response. The term “inhibitor” may also refer to an “antagonist.”

Antibody “effector functions” refer to those biological activities attributable to the Fc region (a native sequence Fe region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-C1q antibody binds specifically to an epitope within a complement C1q protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

An “isolated” nucleic acid molecule is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acids encoding any polypeptides and antibodies herein that exist naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby arc replicated along with the host genome. Moreover, certain vectors are capable of directing the expression of genes to which they are operatively linked. Such vectors are referred to herein as “recombinant expression vectors,” or simply, “expression vectors.” In general, expression vectors useful in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that arc generally known in the art, including, for example, 2′-O-methyl-, 2′-O-allyl-, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or aralkyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “preventing” is art-recognized, and when used in relation to a condition, such as an ocular disease (e.g., glaucoma or age-related macular degeneration, such as AMD, including geographic atrophy) or related symptoms, relative to a patient who does not receive the therapy.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Anti-Complement C1q Antibodies

The anti-C1q antibodies disclosed herein are potent inhibitors of C1q and can be dosed for continuous inhibition of C1q function over any period, and then optionally withdrawn to allow for return of normal C1q function at times when its activity may be important. Results obtained with anti-C1q antibodies disclosed herein in animal studies can be readily carried forward into the clinic with humanized or human antibodies, as well as with fragments and/or derivatives thereof.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the Cl complex (C1qr₂s₂).

The antibodies of this disclosure specifically recognize complement factor C1q and/or C1q in the Cl complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

Anti-C1q antibodies disclosed herein may inhibit C1 complex formation.

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:

C1q, chain A (homo sapiens), Accession No.
Protein Data Base: NP_057075.1; GenBank No.:
NM_015991: >gi|7705753|ref|NP_057075.1|
complement C1q subcomponent subunit A
precursor [Homo sapiens]
(SEQ ID NO: 1)
MEGPRGWLVLCVLAISLASMVTEDLCRAPDGKKGEAGRPGRRGRPGLKGE
QGEPGAPGIRTGIQGLKGDQGEPGPSGNPGKVGYPGPSGPLGARGIPGIK
GTKGSPGNIKDQPRPAFSAIRRNPPMGGNVVIFDTVITNQEEPYQNHSGR
FVCTVPGYYYFTFQVLSQWEICLSIVSSSRGQVRRSLGFCDTTNKGLFQV
VSGGMVLQLQQGDQVWVEKDPKKGHIYQGSEADSVFSGFLIFPSA.
C1q, chain B (homo sapiens), Accession No.
Protein Data Base: NP_000482.3; GenBank No.:
NM_000491.3: >gi|87298828|ref|NP_000482.3|
complement C1q subcomponent subunit B
precursor [Homo sapiens]
(SEQ ID NO: 2)
MMMKIPWGSIPVLMLLLLLGLIDISQAQLSCTGPPAIPGIPGIPGTPGPD
GQPGTPGIKGEKGLPGLAGDHGEFGEKGDPGIPGNPGKVGPKGPMGPKGG
PGAPGAPGPKGESGDYKATQKIAFSATRTINVPLRRDQTIRFDHVITNMN
NNYEPRSGKFTCKVPGLYYFTYHASSRGNLCVNLMRGRERAQKVVTFCDY
AYNTFQVTTGGMVLKLEQGENVFLQATDKNSLLGMEGANSIFSGFLLFPD
MEA.
C1q, chain (homo sapiens), Accession No.
Protein Data Base: NP_001107573.1; GenBank
No.: NM_001114101.1: >gi|166235903|ref|
NP_001107573.1|complement C1q subcomponent
subunit C precursor [Homo sapiens]
(SEQ ID NO: 3)
MDVGPSSLPHLGLKLLLLLLLLPLRGQANTGCYGIPGMPGLPGAPGKDGY
DGLPGPKGEPGIPAIPGIRGPKGQKGEPGLPGHPGKNGPMGPPGMPGVPG
PMGIPGEPGEEGRYKQKFQSVFTVTRQTHQPPAPNSLIRFNAVLTNPQGD
YDTSTGKFTCKVPGLYYFVYHASHTANLCVLLYRSGVKVVTFCGHTSKTN
QVNSGGVLLRLQVGEEVWLAVNDYYDMVGIQGSDSVFSGFLLFPD.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, a chimeric antibody, or a fragment thereof or a derivative thereof. In some embodiments, the antibody is humanized antibody. In some embodiments, the antibody is antibody fragment, such as, a Fab fragment.

All sequences mentioned in the following twenty paragraphs are incorporated by reference from U.S. Pat. No. 9,708,394, which is hereby incorporated by reference for the antibodies and related compositions that it discloses.

### Light Chain and Heavy Chain Variable Domain Sequences of Antibody M1 (Mab1)

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWY
QEKPGKTNKLLIYSGSTLQSGIPSRFSGSGSGTDFT
LTISSLEPEDFAMYYCQQHNEYPLTFGAGTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the MI light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHW
VKQRPGQGLEWIGVIHPNSGSINYNEKFESKATLTV
DKSSSTAYMQLSSLTSEDSAVYYCAGERDSTEVLPM
DYWGQGTSVTVSS.

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIHPNSGS1NYNEKFES (SEQ ID NO:10), and the HVR-H3 of the M1 heavy chain variable domain has the sequence ERDSTEVLPMDY (SEQ ID NO:11).

The nucleic acid sequence encoding the light chain variable domain was determined to be:

(SEQ ID NO: 12)
GATGTCCAGATAACCCAGTCTCCATCTTATCTTGCTGCAT
CTCCTGGAGAAACCATTACTATTAATTGCAGGGCAAGTAA
GAGCATTAACAAATATTTAGCCTGGTATCAAGAGAAACCT
GGGAAAACTAATAAGCTTCTTATCTACTCTGGATCCACTT
TGCAATCTGGAATTCCATCAAGGTTCAGTGGCAGTGGATC
TGGTACAGATTTCACTCTCACCATCAGTAGCCTGGAGCCT
GAAGATTTTGCAATGTATTACTGTCAACAACATAATGAAT
ACCCGCTCACGTTCGGTGCTGGGACCAAGCTGGAGCTGAA
A.

The nucleic acid sequence encoding the heavy chain variable domain was determined to be:

(SEQ ID NO: 13)
CAGGTCCAACTGCAGCAGCCTGGGGCTGAGCTGGTAAAGC
CTGGGGCTTCAGTGAAGTTGTCCTGCAAGTCTTCTGGCTA
CCATTTCACCAGCTACTGGATGCACTGGGTGAAGCAGAGG
CCTGGACAAGGCCTTGAGTGGATTGGAGTGATTCATCCTA
ATAGTGGTAGTATTAACTACAATGAGAAGTTCGAGAGCAA
GGCCACACTGACTGTAGACAAATCCTCCAGCACAGCCTAC
ATGCAACTCAGCAGCCTGACATCTGAGGACTCGGCGGTCT
ATTATTGTGCAGGAGAGAGAGATTCTACGGAGGTTCTCCC
TATGGACTACTGGGGTCAAGGAACCTCAGTCACCGTCTCC
TCA.

### Deposit of Material

The following materials have been deposited according to the Budapest Treaty in the American Type Culture Collection, ATCC Patent Depository, 10801 University Blvd., Manassas, Va. 20110-2209, USA (ATCC):

| | Deposit ATCC | | |
|---|---|---|---|
| Sample ID | Isotype | Date | Accession No. |
| Mouse hybridoma C1qM1 7788-1(M) | IgG1, | Jun. 6, | PTA-120399 |
| 051613 producing anti-C1q antibody | kappa | 2013 | |
| M1 | | | |

The hybridoma cell line producing the M1 antibody (mouse hybridoma C1qM1 7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

Disclosed herein are methods of administering an anti-C1q antibody comprising a light chain variable domain and a heavy chain variable domain. The antibody may bind to at least human C1q, mouse C1q, or rat C1q. The antibody may be a humanized antibody, a chimeric antibody, or a human antibody. The antibody may be a monoclonal antibody, an antibody fragment thereof, and/or an antibody derivative thereof. In some embodiments, the antibody is humanized antibody. In some embodiments, the antibody is antibody fragment, such as, a Fab fragment. The light chain variable domain comprises the HVR-L1, HVR-L2, and HVR-L3 of the monoclonal antibody M1 produced by a hybridoma cell line deposited with Accession Number PTA-120399. The heavy chain variable domain comprises the HVR-H1, HVR-H2, and HVR-H3 of the monoclonal antibody M1 produced by a hybridoma cell line deposited with ATCC Accession Number PTA-120399.

In some embodiments, the amino acid sequence of the light chain variable domain and heavy chain variable domain comprise one or more of SEQ ID NO:5 of HVR-L1, SEQ ID NO:6 of HVR-L2, SEQ ID NO:7 of HVR-L3, SEQ ID NO:9 of HVR-H1, SEQ ID NO:10 of HVR-H2, and SEQ ID NO:11 of HVR-H3. The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

Disclosed herein are methods of administering an anti-C1q antibody, which inhibits the interaction between C1q and an autoantibody. In preferred embodiments, the anti-C1q antibody causes clearance of C1q from the circulation or tissue.

In some embodiments, the anti-C1q antibody of this disclosure inhibits the interaction between C1q and C1s. In some embodiments, the anti-C1q antibody inhibits the interaction between C1q and C1r. In some embodiments the anti-C1q antibody inhibits the interaction between C1q and C1s and between C1q and C1r. In some embodiments, the anti-C1q antibody inhibits the interaction between C1q and another antibody, such as an autoantibody. In preferred embodiments, the anti-C1q antibody causes clearance of C1q from the circulation or tissue. In some embodiments, the anti-C1q antibody inhibits the respective interactions, at a stoichiometry of less than 2.5:1; 2.0:1; 1.5:1; or 1.0:1. In some embodiments, the C1q antibody inhibits an interaction, such as the C1q-C1s interaction, at approximately equimolar concentrations of C1q and the anti-C1q antibody. In other embodiments, the anti-C1q antibody binds to C1q with a stoichiometry of less than 20:1; less than 19.5:1; less than19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1q antibody binds C1q with a binding stoichiometry that ranges from 20:1 to 1.0:1 or less than 1.0:1. In certain embodiments, the anti-C1q antibody binds C1q with a binding stoichiometry that ranges from 6:1 to 1.0:1 or less than1.0:1. In certain embodiments, the anti-C1q antibody binds C1q with a binding stoichiometry that ranges from 2.5:1 to 1.0:1 or less than1.0:1. In some embodiments, the anti-C1q antibody inhibits the interaction between C1q and C1r, or between C1q and C1s, or between C1q and both C1r and C1s. In some embodiments, the anti-C1q antibody inhibits the interaction between C1q and C1r, between C1q and C1s, and/or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain In some embodiments, the anti-C1q antibody binds to the C1q A-chain, the C1q B-chain and/or the C1q C-chain. In some embodiments, the anti-C1q antibody binds to the globular domain of the C1q A-chain, B-chain, and/or C-chain In other embodiments, the anti-C1q antibody binds to the collagen-like domain of the C1q A-chain, the C1q B-chain, and/or the C1q C-chain.

Where antibodies of this disclosure inhibit the interaction between two or more complement factors, such as the interaction of C1q and C1s, or the interaction between C1q and C1r, the interaction occurring in the presence of the antibody may be reduced by at least 10%, at least 20%, at least 30%, at least 40%, at least 50%, at least 60%, at least 70%, at least 80%, at least 90%, at least 95%, or at least 99% relative to a control wherein the antibodies of this disclosure are absent. In some embodiments, antibodies of this disclosure reduces the interaction between two or more complement factors by 50%, 60%, 70%, 80%, 90%, or 100%. In certain embodiments, the interaction occurring in the presence of the antibody is reduced by an amount that ranges from at least 30% to at least 99% relative to a control wherein the antibodies of this disclosure are absent.

In some embodiments, the antibodies of this disclosure inhibit C2 or C4-cleavage by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring C2 or C4-cleavage are well known in the art. The EC₅₀ values for antibodies of this disclosure with respect C2 or C4-cleavage may be less than 3 μg/ml; 2.5 μg/ml; 2.0 μg/ml; 1.5 μg/ml; 1.0 μg/ml; 0.5 μg/ml; 0.25 μg/ml; 0.1 μg/m1; 0.05 μg/ml. In some embodiments, the antibodies of this disclosure inhibit C2 or C4-cleavage at approximately equimolar concentrations of C1q and the respective anti-C1q antibody.

In some embodiments, the antibodies of this disclosure inhibit autoantibody-dependent and complement-dependent cytotoxicity (CDC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. The EC₅₀ values for antibodies of this disclosure with respect to inhibition of autoantibody-dependent and complement-dependent cytotoxicity may be less than 3 μg/ml; 2.5 μg/ml; 2.0 μg/ml; 1.5 μg/ml; 1.01 μg/ml; 0.5 μg/ml; 0.25 μg/ml; 0.1 μg/ml; 0.05 μg/ml.

In some embodiments, the antibodies of this disclosure inhibit complement-dependent cell-mediated cytotoxicity (CDCC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring CDCC are well known in the art. The EC₅₀ values for antibodies of this disclosure with respect CDCC inhibition may be 1 less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 mg/ml. In some embodiments, the antibodies of this disclosure inhibit CDCC but not antibody-dependent cellular cytotoxicity (ADCC).

### Humanized Anti-Complement C1q Antibodies

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat C1q.

All sequences mentioned in the following sixteen paragraphs are incorporated by reference from U.S. patent application Ser. No. 14/933,517, which is hereby incorporated by reference for the antibodies and related compositions that it discloses.

In some embodiments, the human heavy chain constant region is a human IgG4 heavy chain constant region comprising the amino acid sequence of SEQ ID NO:47, or with at least 70%, at least about 75%, at least about 80%, at least about 85%, at least about 90% homology to SEQ ID NO: 47. The human IgG4 heavy chain constant region may comprise an Fc region with one or more modifications and/or amino acid substitutions according to Kabat numbering. In such cases, the Fc region comprises a leucine to glutamate amino acid substitution at position 248, wherein such a substitution inhibits the Fc region from interacting with an Fc receptor. In some embodiments, the Fc region comprises a serine to proline amino acid substitution at position 241, wherein such a substitution prevents arm switching in the antibody.

The amino acid sequence of human IgG4 (S241P L248E) heavy chain constant domain is:

(SEQ ID NO: 47)
ASTKGPSVFPLAPCSRSTSESTAALGCLVKDYFPEPVTVS
WNSGALTSGVHTFPAVLQSSGLYSLSSVVTVPSSSLGTKT
YTCNVDHKPSNTKVDKRVESKYGPPCPPCPAPEFEGGPSV
FLFPPKPKDTLMISRTPEVTCVVVDVSQEDPEVQFNWYVD
GVEVHNAKTKPREEQFNSTYRVVSVLTVLHQDWLNGKEYK
CKVSNKGLPSSIEKTISKAKGQPREPQVYTLPPSQEEMTK
NQVSLTCLVKGFYPSDIAVEWESNGQPENNYKTTPPVLDS
DGSFFLYSRLTVDKSRWQEGNVFSCSVMHEALHNHYTQKS
LSLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38.

The amino acid sequence of heavy chain variable domain variant 1 (VH1) is:

(SEQ ID NO: 31)
QVQLVQSGAELKKPGASVKVSCKSSGYHFTSYWMHWVKQA
PGQGLEWIGVIHPNSGSINYNEKFESKATITVDKSTSTAY
MQLSSLTSEDSAVYYCAGERDSTEVLPMDYWGQGTSVTVS
S.
The hyper variable regions (HVRs) of VH1
are depicted in bolded and underlined text.

The amino acid sequence of heavy chain variable domain variant 2 (VH2) is:

(SEQ ID NO: 32)
QVQLVQSGAELKKPGASVKVSCKSSGYHFTSYWMHWVKQA
PGQGLEWIGVIHPNSGSINYNEKFESRATITVDKSTSTAY
MELSSLRSEDTAVYYCAGERDSTEVLPMDYWGQGTTVTVS
S.
The hyper variable regions (HVRs) of VH2
are depicted in bolded and underlined text.

The amino acid sequence of heavy chain variable domain variant 3 (VH3) is:

(SEQ ID NO: 33)
QVQLVQSGAELKKPGASVKVSCKSSGYHFTSYWMHWVKQA
PGQGLEWIGVIHPNSGSINYNEKFESRVTITVDKSTSTAY
MELSSLRSEDTAVYYCAGERDSTEVLPMDYWGQGTTVTVS
S.
The hyper variable regions (HVRs) of VH3
are depicted in bolded and underlined text.

The amino acid sequence of heavy chain variable domain variant 4 (VH4) is:

(SEQ ID NO: 34)
QVQLVQSGAELKKPGASVKVSCKSSGYHFTSYWMHWVRQAP
GQGLEWIGVIHPNSGSINYNEKFESRVTITVDKSTSTAYME
LSSLRSEDTAVYYCAGERDSTEVLPMDYWGQGTTVTVSS.
The hyper variable regions (HVRs) of VH4
are depicted in bolded and underlined text.

The amino acid sequence of kappa light chain variable domain variant 1 (Vκ1) is:

(SEQ ID NO: 35)
DVQITQSPSYLAASLGERATINCRASKSINKYLAWYQQKP
GKTNKLLIYSGSTLQSGIPARFSGSGSGTDFTLTISSLEP
EDFAMYYCQQHNEYPLTFGQGTKLEIK.
The hyper variable regions (HVRs) of VK1
are depicted in bolded and underlined text.

The amino acid sequence of kappa light chain variable domain variant 2 (Vκ2) is:

(SEQ ID NO: 36)
DVQITQSPSSLSASLGERATINCRASKSINKYLAWYQQKP
GKANKLLIYSGSTLQSGIPARFSGSGSGTDFTLTISSLEP
EDFAMYYCQQHNEYPLTFGQGTKLEIK.
The hyper variable regions (HVRs) of VK2
are depicted in bolded and underlined text.

The amino acid sequence of kappa light chain variable domain variant 3 (Vκ3) is:

(SEQ ID NO: 37)
DVQITQSPSSLSASLGERATINCRASKSINKYLAWYQQKP
GKAPKLLIYSGSTLQSGIPARFSGSGSGTDFTLTISSLEP
EDFAMYYCQQHNEYPLTFGQGTKLEIK.
The hyper variable regions (HVRs) of VK3
are depicted in bolded and underlined text.

The amino acid sequence of kappa light chain variable domain variant 4 (Vκ4) is:

(SEQ ID NO: 38)
DIQLTQSPSSLSASLGERATINCRASKSINKYLAWYQQKP
GKAPKLLIYSGSTLQSGIPARFSGSGSGTDFTLTISSLEP
EDFAMYYCQQHNEYPLTFGQGTKLEIK.
The hyper variable regions (HVRs) of VK4
are depicted in bolded and underlined text.

The antibody may comprise a light chain variable domain amino acid sequence that is at least 85%, 90%, or 95% identical to SEQ ID NO:35-38 while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:31-34 while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

In some embodiments, the antibody comprises a light chain variable domain amino acid sequence of SEQ ID NO: 35 and a heavy chain variable domain amino acid sequence of SEQ ID NO: 31. In some embodiments, the antibody comprises a light chain variable domain amino acid sequence of SEQ ID NO: 36 and a heavy chain variable domain amino acid sequence of SEQ ID NO: 32. In some embodiments, the antibody comprises a light chain variable domain amino acid sequence of SEQ ID NO: 37 and a heavy chain variable domain amino acid sequence of SEQ ID NO: 33. In some embodiments, the antibody comprises a light chain variable domain amino acid sequence of SEQ ID NO: 38 and a heavy chain variable domain amino acid sequence of SEQ ID NO: 34.

In some embodiments, humanized anti-C1q antibodies of the present disclosure include a heavy chain variable region that contains an Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or IgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a positions corresponding to position 248 inhibits the Fc region from interacting with an Fc receptor. In some embodiments, the amino acid substitution at position 248 or a positions corresponding to position 248 is a leucine to glutamate amino acid substitution. In some embodiments, the amino acid substitution at position 241 or a positions corresponding to position 241 prevents arm switching in the antibody. In some embodiments, the amino acid substitution at position 241 or a positions corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

### Anti-C1q Fab Fragment

Before the advent of recombinant DNA technology, proteolytic enzymes (proteases) that cleave polypeptide sequences have been used to dissect the structure of antibody molecules and to determine which parts of the molecule are responsible for its various functions. Limited digestion with the protease papain cleaves antibody molecules into three fragments. Two fragments, known as Fab fragments, are identical and contain the antigen-binding activity. The Fab fragments correspond to the two identical arms of the antibody molecule, each of which consists of a complete light chain paired with the V_{H} and C_{H}1 domains of a heavy chain. The other fragment contains no antigen binding activity but was originally observed to crystallize readily, and for this reason was named the Fc fragment (Fragment crystallizable). When Fab molecules were compared to IgG molecules, it was found that Fab are superior to IgG for certain in vivo applications due to their higher mobility and tissue penetration capability, their reduced circulatory half-life, their ability to bind antigen monovalently without mediating antibody effector functions, and their lower immunogenicity.

The Fab molecule is an artificial ˜50-kDa fragment of the Ig molecule with a heavy chain shortened by constant domains C_{H}2 and C_{H}3. Two heterophilic (V_{L}-V_{H} and C_{L}-C_{H}1) domain interactions underlie the two-chain structure of the Fab molecule, which is further stabilized by a disulfide bridge between C_{L} and C_{H}1. Fab and IgG have identical antigen binding sites formed by six complementarity-determining regions (CDRs), three each from V_{L} and V_{H} (LCDR1, LCDR2, LCDR3 and HCDR1, HCDR2, HCDR3). The CDRs define the hypervariable antigen binding site of antibodies. The highest sequence variation is found in LCDR3 and HCDR3, which in natural immune systems are generated by the rearrangement of V_{L} and J_{L} genes or V_{H}, D_{H} and J_{H} genes, respectively. LCDR3 and HCDR3 typically form the core of the antigen binding site. The conserved regions that connect and display the six CDRs are referred to as framework regions. In the three-dimensional structure of the variable domain, the framework regions form a sandwich of two opposing antiparallel β-sheets that are linked by hypervariable CDR loops on the outside and by a conserved disulfide bridge on the inside. This unique combination of stability and versatility of the antigen binding site of Fab and IgG underlie its success in clinical practice for the diagnosis, monitoring, prevention, and treatment of disease.

All anti-C1q antibody Fab fragment sequences are incorporated by reference from U.S. patent application Ser. No. 15/360,549, which is hereby incorporated by reference for the antibodies and related compositions that it discloses.

In certain embodiments, the present disclosure provides an anti-C1q antibody Fab fragment that binds to a C1q protein comprising a heavy (V_{H}/C_{H}1) and light chain (V_{L}/C_{L}), wherein the anti-C1q antibody Fab fragment has six complementarity determining regions (CDRs), three each from V_{L} and V_{H} (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence:

(SEQ ID NO: 39)
QVQLVQSGAELKKPGASVKVSCKSSGYHFTSYWMHWVKQA
PGQGLEWIGVIHPNSGSINYNEKFESRVTITVDKSTSTAY
MELSSLRSEDTAVYYCAGERDSTEVLPMDYWGQGTTVTVS
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDKTHT

The complementarity determining regions (CDRs) of SEQ ID NO:39 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKP
GKAPKLLIYSGSTLQSGIPARFSGSGSGTDFTLTISSLEP
EDFAMYYCQQHNEYPLTFGQGTKLEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC

The complementarity determining regions (CDRs) of SEQ ID NO:40 are depicted in bolded and underlined text.

### Nucleic Acids, Vectors and Host Cells

Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-C1q antibody. In some embodiments, one or more vectors (e.g., expression vectors) containing such nucleic acids are provided. A host cell containing such nucleic acid may also be provided. The host cell may contain (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the V_{L}/C_{L} of the antibody and a second vector containing a nucleic acid that encodes an amino acid sequence containing the V_{H}/C_{H}1 of the antibody. In some embodiments, the host cell is eukaryotic, e.g., a Chinese Hamster Ovary (CHO) cell or lymphoid cell (e.g., Y0, NS0, Sp20 cell). In some embodiments, the host cell is a bacterium such as E. coli.

Methods of making an anti-C1q antibody are disclosed herein. The method includes culturing a host cell of the present disclosure containing a nucleic acid encoding the anti-C1q antibody, under conditions suitable for expression of the antibody. In some embodiments, the antibody is subsequently recovered from the host cell (or host cell culture medium).

For recombinant production of a humanized anti-C1q antibody of the present disclosure, a nucleic acid encoding the antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, Co1E1, pCR1, RP4, phage DNAs, and shuttle vectors such as pSA3 and pAT28. These and many other cloning vectors are available from commercial vendors such as BioRad, Stratagene, and Invitrogen.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-C1q antibody of the present disclosure.

Suitable host cells for cloning or expression of antibody-encoding vectors include prokaryotic or eukaryotic cells. For example, an anti-C1q antibody of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B.K.C. Lo, ed., Humana Press, Totowa, NJ, 2003), pp. 245-254, describing expression of antibody fragments in E. coli.). In other embodiments, the antibody of the present disclosure may be produced in eukaryotic cells, e.g., a Chinese Hamster Ovary (CHO) cell or lymphoid cell (e.g., Y0, NS0, Sp20 cell) (e.g., U.S. patent application Ser. No. 14/269,950, U.S. Pat. No. 8,981,071, Eur J Biochem. 1991 Jan 1; 195 (1):235-42). After expression, the antibody may be isolated from the bacterial cell paste in a soluble fraction and can be further purified.

### Pharmaceutical Compositions and Administration

The anti-C1q antibody (e.g., FabA) of the present disclosure may be administered in the form of pharmaceutical compositions.

Therapeutic formulations of an antibody, antibody fragments and/or antibody derivatives of the disclosure may be prepared for storage by mixing the antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Lipofections or liposomes may also be used to deliver an antibody or antibody fragment, or antibody derivative into a cell, wherein the epitope or smallest fragment which specifically binds to the binding domain of the target protein is preferred.

The antibody may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for administration may be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

The antibodies, antibody fragments and/or antibody derivatives and compositions of the present disclosure are typically administered by an intravitreal administration.

Pharmaceutical compositions may also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation may include other carriers, adjuvants, or non-toxic, nontherapeutic, non-immunogenic stabilizers, excipients and the like. The compositions may also include additional substances to approximate physiological conditions, such as pH adjusting and buffering agents, toxicity adjusting agents, wetting agents and detergents.

The composition may also include any of a variety of stabilizing agents, such as an antioxidant for example. When the pharmaceutical composition includes a polypeptide, the polypeptide may be complexed with various well-known compounds that enhance the in vivo stability of the polypeptide, or otherwise enhance its pharmacological properties (e.g., increase the half-life of the polypeptide, reduce its toxicity, enhance other pharmacokinetic and/or pharmacodynamic characteristics, or enhance solubility or uptake). Examples of such modifications or complexing agents include sulfate, gluconate, citrate and phosphate. The polypeptides of a composition may also be complexed with molecules that enhance their in vivo attributes. Such molecules include, for example, carbohydrates, polyamines, amino acids, other peptides, ions (e.g., sodium, potassium, calcium, magnesium, manganese), and lipids. Further guidance regarding formulations that are suitable for various types of administration may be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredient may be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it may be expressed as the ratio LD50/ED50. Compounds that exhibit large therapeutic indices are preferred.

The data obtained from cell culture and/or animal studies and/or human clinical trials may be used in formulating a range of dosages for humans. The dosage of the active ingredient typically lines within a range of circulating concentrations that include the ED50 with low toxicity. The dosage may vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for parenteral use are usually sterile. To the extent that a given compound must be synthesized prior to use, the resulting product is typically substantially free of any potentially toxic agents, particularly any endotoxins, which may be present during the synthesis or purification process. Compositions for parental administration are also typically substantially isotonic and made under GMP conditions.

The compositions of the disclosure may be administered using any medically appropriate procedure, e.g., intravitreal injection.

### Methods of Treatment

The present disclosure is generally directed to compositions and methods of preventing, reducing risk of developing, or treating an ocular disease (e.g., glaucoma or age-related macular degeneration, such as AMD, including geographic atrophy) in a human patient. Such methods include administering to the patient a composition comprising about 1 mg to about 10 mg (e.g., about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of the anti-C1q antibody) of an anti-C1q antibody via an intravitreal injection. Such methods also include administering to the patient a composition comprising about 1 mg to about 10 mg (e.g., about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of the anti-C1q antibody) of an anti-C1q antibody via an intravitreal injection, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7; and a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. The administered composition may comprise about 1 mg to about 5 mg of the anti-C1q antibody. The administered composition may comprise about 1 mg to about 2.5 mg, about 2.5 mg to about 5 mg, about 5 mg to about 7.5 mg, or about 7.5 mg to about 10 mg of the anti-C1q antibody. The administered composition may comprise about 5 mg of the anti-C1q antibody. The administered composition may comprise about 10 mg of the anti-C1q antibody. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the heavy chain variable domain comprising an amino acid sequence selected from SEQ ID NO: 8 and 31-34. In some embodiments, the antibody comprises a light chain variable domain comprising an amino acid sequence with at least about 95% homology to the amino acid sequence selected from SEQ ID NO: 4 and 35-38, and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, and a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence selected from SEQ ID NO: 4 and 35-38, and a heavy chain variable domain comprising an amino acid sequence selected from SEQ ID NO: 8 and 31-34. In some embodiments, the antibody may be a monoclonal antibody, a humanized antibody, a human antibody, a chimeric antibody, an antibody fragment, or antibody derivative thereof. The antibody fragment may be a Fab fragment, a Fab′ fragment, a F(ab′)2 fragment, a Fv fragment, a diabody, or a single chain antibody molecule. In some embodiments, the Fab fragment comprises a heavy chain Fab fragment of SEQ ID NO: 39 and a light chain Fab fragment of SEQ ID NO: 40.

In some embodiments, the antibody is administered once a week, once every other week, once every three weeks, once a month, once every 4 weeks, once every six weeks, once every 8 weeks, once every other month, once every 10 weeks, once every 12 weeks, once every three months, or once every 4 months. In some embodiments, the antibody is administered for at least 6 months, at least 7 months, at least 8 months, at least 9 months, at least 10 months, at least 11 months, or at least 12 months.

In some embodiments, the ocular disease is glaucoma or age-related macular degeneration, such as geographic atrophy.

In certain preferred embodiments, FabA is administered at a dose of 2.5 mg/eye once every month, once every 4 weeks, once every 6 weeks, or once every other month as an IVT injection.

In certain preferred embodiments, FabA is administered at a dose of 5 mg/eye once every month, once every 4 weeks, once every 6 weeks, or every other month as an IVT injection.

In certain preferred embodiments, FabA is administered at a dose of 5 mg/eye once every month, once every 4 weeks, once every 6 weeks, or once every other month as an IVT injection.

In certain preferred embodiments, FabA is administered at a dose of 10 mg/eye once every month, once every 4 weeks, once every 6 weeks, or every other month as an IVT injection.

Injection of FabA is completed by a physician qualified by training and experience to perform IVT injections, using aseptic technique.

The anti-C1q antibody may inhibit the interaction between C1q and an autoantibody or between C1q and Cir, or between C1q and Cis, or may promote clearance of C1q from circulation or a tissue. In some embodiments, the anti-C1q antibody has a dissociation constant (K_{D}) that ranges from 100 nM to 0.005 nM or less than 0.005 nM. In some embodiments, the anti-C1q antibody binds C1q with a binding stoichiometry that ranges from 20:1 to 1.0:1 or less than 1.0:1, a binding stoichiometry that ranges from 6:1 to 1.0:1 or less than 1.0:1, or a binding stoichiometry that ranges from 2.5:1 to 1.0:1 or less than 1.0:1.

The methods inhibit a biological activity of C1q. For example, (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to B-amyloid, or (10) C1q binding to calreticulin. In other embodiments, the biological activity of C1q is (1) activation of the classical complement activation pathway, (2) reduction in lysis and/or reduction in C3 deposition, (3) activation of antibody and complement dependent cytotoxicity, (4) CH50 hemolysis, (5) a reduction in red blood cell lysis, (6) a reduction in red blood cell phagocytosis, (7) a reduction in dendritic cell infiltration, (8) inhibition of complement-mediated red blood cell lysis, (9) a reduction in lymphocyte infiltration, (10) a reduction in macrophage infiltration, (11) a reduction in antibody deposition, (12) a reduction in neutrophil infiltration, (13) a reduction in platelet phagocytosis, (14) a reduction in platelet lysis, (15) an improvement in transplant graft survival, (16) a reduction in macrophage mediated phagocytosis, (17) a reduction in autoantibody mediated complement activation, (18) a reduction in red blood cell destruction due to transfusion reactions, (19) a reduction in red blood cell lysis due to alloantibodies, (20) a reduction in hemolysis due to transfusion reactions, (21) a reduction in alloantibody mediated platelet lysis, (22) an improvement in anemia, (23) a reduction in eosinophilia, (24) a reduction in C3 deposition on red blood cells (e.g., a reduction of deposition of C3b, iC3b, etc., on RBCs), (25) a reduction in C3 deposition on platelets (e.g., a reduction of deposition of C3b, iC3b, etc., on platelets), (26) reduction in anaphylatoxin production, (27) a reduction in autoantibody mediated blister formation, (28) a reduction in autoantibody induced erythematosus, (29) a reduction in red blood cell destruction due to transfusion reactions, (30) a reduction in platelet lysis due to transfusion reactions, (31) a reduction in mast cell activation, (32) a reduction in mast cell histamine release, (33) a reduction in vascular permeability, (34) a reduction in complement deposition on transplant graft endothelium, (35) B-cell antibody production, (36) dendritic cell maturation, (37) T-cell proliferation, (38) cytokine production, (39) microglia activation, (40) Arthus reaction, (41) a reduction of anaphylatoxin generation in transplant graft endothelium, or (42) activation of complement receptor 3 (CR3/C3) expressing cells.

In some embodiments, CH50 hemolysis comprises human CH50 hemolysis. The antibody may be capable of neutralizing from at least about 50%, to about 100% of human CH50 hemolysis. The antibody may be capable of neutralizing about 50%, about 60%, about 70%, about 80%, about 90%, about 100% of human CH50 hemolysis. The antibody may be capable of neutralizing at least 50% of CH50 hemolysis at a dose of less than 150 ng/ml, less than 100 ng/ml, less than 50 ng/ml, or less than 20 ng/ml.

In some embodiments, the antibody is a monoclonal antibody, a polyclonal antibody, a recombinant antibody, a humanized antibody, a human antibody, a chimeric antibody, a monovalent antibody, a multispecific antibody, or an antibody fragment, or antibody derivative thereof. In some embodiments, the antibody is humanized antibody. In some embodiments, the antibody is antibody fragment, such as, a Fab fragment. Examples of an antibody fragment are a Fab fragment, a Fab′ fragment, a F(ab′)2 fragment, a Fv fragment, a diabody, and a single chain antibody molecule.

It is contemplated that compositions may be obtained and used under the guidance of a physician for in vivo use. The dosage of the therapeutic formulation may vary widely, depending upon the nature of the disease, the frequency of administration, the manner of administration, the clearance of the agent from the host, and the like.

As used herein, “chronically administered,” “chronic treatment,” “treating chronically,” or similar grammatical variations thereof refer to a treatment regimen that is employed to maintain a certain threshold concentration of a therapeutic agent in the eye of a patient in order to completely or substantially suppress systemic complement activity in the patient over a prolonged period of time. Accordingly, a patient chronically treated with anti-C1q antibody may be treated for a period of time that is greater than or equal to 2 weeks (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52 weeks; 1, 2, 3, 4, 5, 6, 7, 8, 9, 10, 11, or 12 months; or 1, 1.5, 2, 2.5, 3, 3.5, 4, 4.5, 5, 5.5, 6, 6.5, 7, 7.5, 8, 8.5, 9, 9.5, 10, 10.5, or 12 years or for the remainder of the patient's life) with the antibody in an amount and with a dosing frequency that are sufficient to maintain a concentration of the antibody in the patient's eye that inhibits or substantially inhibits systemic complement activity in the patient. In some embodiments, the antibody may be chronically administered to a patient in need thereof in an amount and with a frequency that are effective to maintain serum hemolytic activity at less than or equal to 20% (e.g., 19, 18, 17, 16, 15, 14, 13, 12, 11, 10, 9, 8, 7, 6, or even below 5%). In some embodiments, the antibody may be administered to a patient in an amount and with a frequency that are effective to maintain serum lactate dehydrogenase (LDH) levels at within at least 20% (e.g., 19, 18, 17, 16, 15, 14, 13, 12, 11, 10, 9, 8, 7, 6, or even below 5%) the normal range for LDH.

Therapeutic agents, e.g., anti-C1q antibodies, can be incorporated into a variety of formulations for therapeutic administration by combination with appropriate pharmaceutically acceptable carriers or diluents.

### EXAMPLES

### Example 1

### Evaluating FabA in Nonclinical Studies

FabA Drug Product is a sterile, isotonic liquid for IVT injection.

FabA is provided as sterile, single-use vials for IVT injection.

An extensive series of in vitro and in vivo pharmacology studies have been conducted with FabA.

The antibody Mab1, Mab1-Fab, and Mab2, were active in an acute mouse model of glaucoma, protecting against retinal ganglion cell and/or nerve fiber loss. In a photo-oxidative light-induced damage model in mouse, Mab1 administered intravitreally protected against photoreceptor cell loss and retinal functional connectivity in the eye.

The FabA GLP studies consist of a single-dose rat ocular toxicology study, and three repeat-dose cynomolgus monkey ocular toxicology studies. The route of administration for the toxicology studies was IVT injection. In the single dose and two dose (once monthly) IVT GLP studies, FabA has shown no evidence of adverse ocular toxicity with a No-Observed-Adverse-Effect-Level (NOAEL) of 5 mg/eye (equivalent to 10 mg human dose) once monthly in cynomolgus monkeys, and 0.05 mg/eye (equivalent to 10 mg human dose) in the single dose rat study. In the 26-week chronic ocular toxicology study in cynomolgus monkeys, adverse ocular changes were associated with the double injection procedure and/or determined to be anti-drug antibody (ADA)-mediated and not a direct effect of FabA IVT administration.

Pharmacokinetic evaluations of FabA in rat and cynomolgus monkey serum and vitreous were conducted. C1q levels were measured in the vitreous as a PD marker for FabA inhibition of C1q in monkeys. The PK/PD and TK/PD studies in monkeys demonstrated a robust ocular PD effect consistent with the level of FabA drug exposure in the vitreous.

### Binding and Affinity of FabA and Precursor Molecules for Human C1q

C1q binding affinity of FabA and precursor molecules was also examined by ELISA. All molecules (Mab2-Fab, FabA, and Mab2, Mab1, and Mab1-Fab) showed affinity for human C1q with half maximal effective concentration (EC50) in the range of 2.2-4.9 ng/mL (20-95 pM). The EC50 for FabA binding to C1q is 2.5 ng/mL.

### Effect on IgM-Mediated Red Blood Cell Hemolysis

The activity of FabA, Mab2, and Mab2-Fab to functionally-inhibit classical complement-dependent hemolysis of IgM opsonized-RBCs in human serum was measured (FIG. 3). The three molecules exhibit nearly identical potency, consistent with their equivalent binding affinities. The half maximal inhibitory concentration (IC50) of FabA inhibition of IgM-coated RBC hemolysis is 0.62 μg/mL (˜12 nM).

### In vivo Pharmacology Studies

### Anti-C1q Antibody Treatment Prevents Optic Nerve Damage in an Acute Mouse Model of Glaucoma

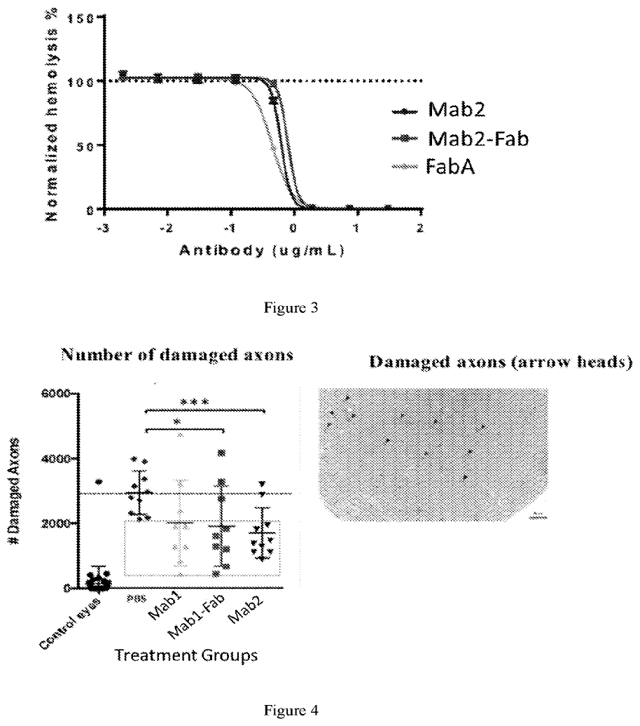

In mice, injection of polystyrene beads into the anterior chamber of the eye results in acute elevation of IOP, loss of the retinal ganglion cells, and optic nerve damage over a period of 2 weeks. Mab1, Mab1-Fab and Mab2 were administered intravitreally into mice on the day before and 7 days after IOP elevation. 2 μL of 10 mg/mL antibody or saline was administered at each time point. Based on a vitreal volume of 5-10 μL in mouse eye, the concentration of antibody was 2000-4000 μg/mL. Optic nerves were collected 2 weeks following the injury and the number of intact and damaged axons was quantified. Anti-C1q antibody treatment led to protection against RGC loss and/or retinal nerve fiber damage in this induced mouse model of glaucoma (FIG. 4).

### Anti-C1q Antibody Treatment Protects Against Photoreceptor Cell Damage in a Photo-Oxidative Light-Induced Damage Model

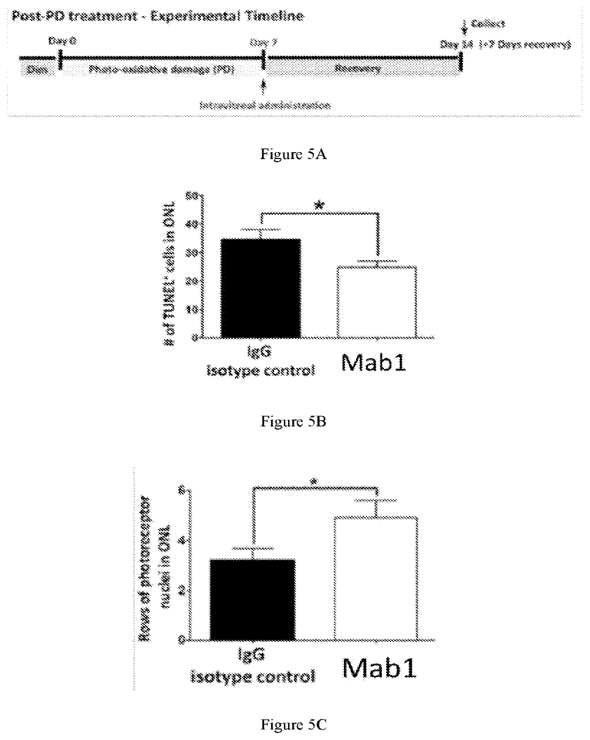

Photo-oxidative damage resulted in retinal photoreceptor loss when mice were exposed to 100 Klux of natural white LED for 1-7 days. In this model, there was time dependent increase in C1qa gene expression over 3-7 days which correlated with photoreceptor cell death and microglia/macrophage recruitment. C1qa−/− mice displayed less photoreceptor cell death, reduced microglia/macrophage recruitment to the photoreceptor lesion, and higher visual function at 14 days after induction of photodamage but not at 7 days. IVT administration of Mab1 antibody on Day 7 after photodamage reduced photoreceptor cell loss and maintained retinal function as measured by electroretinogram (FIG. 5). Mice were administered 11.1.L of 7.5 mg/mL antibody, which is equivalent to 750-1500 ug/mL concentration in the vitreous. In contrast, systemic delivery of Mab1 at 100 mg/kg on Day 0, 4, and 8 had no effect of photoreceptor loss or function. Retinal C1q was mainly expressed by subretinal microglia/macrophages located in the outer retina in early AMD and in mouse retinas. Thus, protection with anti-C1q antibody suggests a clear role of C1q in initiation of photoreceptor damage and the classical complement cascade in the pathogenesis of GA in human disease.

### Safety Pharmacology

Systemic exposure following chronic IVT dosing in the 26-week cynomolgus monkeys did not exceed 86.3 ng/mL, while systemic exposure of Mab2 with the same CDR with once weekly IV dosing in a 26-week cynomolgus monkey study exceeded 1 mg/mL at the NOAEL of 200 mg/kg.

Thus, the safety pharmacology endpoints for the full-length antibody, Mab2, following IV administration up to 200 mg/kg weekly in a 4-week repeat-dose GLP toxicity study in monkeys and up to 200 mg/kg weekly in a 26-week repeat-dose toxicity study in monkeys, in which there was no evidence of a treatment-related effect on cardiovascular, respiratory, or neurologic endpoints support the systemic safety of FabA administered IVT.

Additionally, the safety pharmacology endpoints for FabA, following SC administration up to 20 mg/kg daily in a 4-week repeat-dose GLP toxicity study in monkeys where there was no evidence of a treatment-related effect on cardiovascular, respiratory, or neurologic endpoints support the systemic safety of FabA administered IVT.

### Pharmacokinetics in Animals

Nonclinical studies designed to characterize the PK, TK, and PD of FabA were conducted in rats and in cynomolgus monkeys. These studies include single dose IVT PK studies in rats and cynomolgus monkeys, and repeat-dose TK/PD studies in the cynomolgus monkey with FabA. More extensive TK/PD studies were performed in the monkey, and there was no ocular toxicity in either the rat or monkey single dose studies.

### Pharmacokinetic/Toxicokinetic/Pharmacodynamic Analyses

### Pharmacokinetics of FabA in the Vitreous

Following single bilateral IVT administration of FabA at a dose of 0.01 mg/eye (equivalent to 2 mg human dose) or 0.05 mg/eye (equivalent to 10 mg human dose) to rats, the drug was eliminated from the vitreous relatively quickly, consistent with a half-life of approximately 12 hours at both dose levels. In cynomolgus monkeys, also receiving bilateral IVT FabA, the drug was distributed from the vitreous more slowly, when compared to rats, with a half-life of approximately 3 days for both the 1 mg/eye (equivalent to 2 mg human dose) and the 5 mg/eye (equivalent to 10 mg human dose) dose groups.

In both species, FabA IVT PK was dose linear. Data from the ocular toxicology studies in cynomolgus monkeys, where FabA was given twice over a 28-day period at doses of 1.0 mg/eye (equivalent to 2 mg human dose), 2.5 mg/eye (equivalent to 5 mg human dose), or 5.0 mg/eye (equivalent to 10 mg human dose), indicate that vitreous concentrations at the time of sacrifice (i.e., 15 and 30 days after the second dose), were generally consistent with data from the single dose IVT administration.

In the 26-week chronic ocular toxicology study in cynomolgus monkeys, where FabA was dosed IVT 2.5 mg/eye monthly (equivalent to 5 mg human dose), 5 mg/eye monthly (equivalent to 10 mg human dose), or 5 mg/eye biweekly (both 5 mg/eye doses were subsequently reduced to 2.5 mg/eye and are referred to as 5/2.5 mg/eye) vitreous humor FabA concentrations were quantifiable on Day 184 in all animals that received FabA through Day 169, and were below the quantification limit (BQL) in all animals on Day 242/243 after the 10-week dose-free recovery period. Vitreous humor FabA concentrations showed high variability with no clear differences or trends between dose groups or sexes.

### Pharmacokinetics of FabA in the Serum

Following single IVT administration, serum concentrations were much lower than in the vitreous with a Cₘₐₓ serum/Cₘₐₓ vitreous of ˜0.003 in rats and 0.000001 in cynomolgus monkeys. In cynomolgus monkeys who received bilateral FabA IVT twice over a 28-day period, the serum concentrations were low and the highest mean peak concentration (Cₘₐₓ) was 10.1 ng/mL, which was observed after administration of the second IVT dose of 5 mg/eye (equivalent to 10 mg human dose). As FabA distributes from the vitreous into the serum compar intent, FabA can either bind to C1q, or remain in its free form and be quantifiable with the assay, resulting in low FabA serum concentrations which were not quantifiable (i.e., <1.25 ng/mL) in the 1 mg/eye group, and a mean Cₘₐₓ of 3.3 and 10.1 ng/mL for the 2.5 and 5.0 mg/eye, respectively.

In contrast, following IV administration of FabA at a dose of 10 mg/kg, FabA maximum concentrations were 13800 and 17000 ng/mL in the 2 cynomolgus monkeys tested and the concentrations declined very rapidly afterwards, consistent with a half-life of approximately 2 hours, as expected for a Fab fragment.

When comparing the serum FabA concentrations after bilateral IVT administration (5 mg/eye) twice over a 28-day period to those obtained after systemic IV administration of Mab2 at a dose of 200 mg/kg once weekly for 4 weeks, the FabA serum exposure were considerably lower (FabA/Mab2 Cmax ratio of 0.00000701).

In the 26-week chronic ocular toxicology study in cynomolgus monkeys, where FabA was dosed IVT 2.5 mg/eye monthly, 5/2.5 mg/eye monthly, or 5/2.5 mg/eye biweekly systemic exposure to FabA in serum was low, consistent with the local route of administration. Serum concentrations of FabA on Day 85 did not exceed 86.3 ng/mL and on Day 169 after the last dose was adminstered, FabA serum concentrations did not exceed 60.8 ng/mL. Maximum serum FabA concentrations were observed at 24 to 48 hour postdose across dose levels/regimens and evaluation days. The half-life (T1/2) values for FabA in serum were calculable/reportable only in a few instances in animals in the 5/2.5 mg/eye biweekly group and ranged from 49.9 to 143 hours across all evaluation days, likely representing distribution from the ocular space into the serum. There was little accumulation of FabA in serum with repeated monthly IVT dosing at 2.5 mg/eye. However, there were increasingly more calculable FabA serum concentrations in this group on each subsequent evaluation day after Day 1. Accumulation could not be determined from Day 1 in any other groups due to the changes in dose levels after Day

57. Day 169/Day 85 area under the curve to time “t” (AUC[0-t]) ratios ranged from 0.0407 to 0.664 in 5/2.5 mg/eye once monthly males and females, and ranged from 0.132 to 7.15 in 5/2.5 mg/eye biweekly males and females. When comparing FabA and Mab2 mean sex combined systemic exposures after chronic 26-week dosing in cynomolgus monkeys, FabA exposure AUCO-t (1,230 hr*ng/mL or 1.23 hr*μg/mL) after bilateral IVT administration at 5/2.5 mg/eye biweekly compared to the 200 mg/kg AUC0-t (3,150,000 hr*μg/mL) obtained after once weekly systemic IV administration of Mab2, the FabA serum exposures were considerably lower (FabA/Mab2 Cₘₐₓ ratio of 0.000000073, AUCO-t ratio of 0.00000039).

### Pharmacodynamics of Ocular C1q

In control animals, mean vitreous free C1q concentration was 40.3 ng/mL while in the vitreous of cynomolgus monkeys receiving a single FabA IVT dose of either 1 mg/eye (equivalent to 2 mg human dose) or 5 mg/eye (equivalent to 10 mg human dose), free C1q levels were below the detection limit (<1.953 ng/mL) for the study duration (30 days), indicating complete C1q suppression. In the monkeys who had received FabA every 28 days for a total of 2 doses, C1q remained suppressed for 15 days after administration of the second dose at all 3 dose levels (i.e., 1, 2.5 and 5 mg/eye q 28 days×2 doses). Thirty days after administration of the second FabA dose, C1q remained below the detection limit in some, but not all eyes.

Fifteen days after administration of the second 5 mg/eye IVT, >80% of C1q was bound to FabA in the retina, choroid and optic nerve head. Thirty days after the second administration of 5 mg/eye, C1q remained suppressed in the retina and choroid only.

In the 26-week chronic ocular toxicology study in cynomolgus monkeys, where FabA was dosed IVT 2.5 mg/eye monthly, 5/2.5 mg/eye monthly, or 5/2.5 mg/eye biweekly, at the terminal necropsy, there was a reduction of vitreous humor C1q levels in all groups that received FabA; and at the terminal and recovery necropsies, animals with dose holidays and/or after the 10-week dose-free recovery period, respectively, vitreous humor C1q levels recovered and were comparable to the control group.

### Serum C1q and Serum Hemolysis Inhibition

Following bilateral IVT administration of 5 mg/eye (equivalent to 10 mg human dose) to cynomolgus monkeys, C1q-dependent serum hemolysis was inhibited by ˜50-80%, which lasted approximately 24-48 hours after administration of the first IVT dose, and up to 96 hours following administration of the second FabA dose, returning towards baseline afterwards.

After a single IV administration of 10 mg/kg to cynomolgus monkeys, maximum C1q-dependent serum hemolysis inhibition was reached at 1 hour. Maximum inhibition was maintained for ˜24 hours and returned to baseline 120 hours after FabA administration. Serum free C1q also declined rapidly but had not returned to baseline value by 120 hours, which suggests that some FabA remained bound to circulating C1q over this time frame.

### Toxicology

The safety of FabA is supported by a comprehensive nonclinical ocular toxicology program designed to support the use of FabA for IVT administration in clinical trials. Initial single dose studies were performed in the rat and cynomolgus monkey with FabA and no ocular toxicity was observed in either of these species. Based on the similar findings in rats and monkeys, and the in vitro pharmacology data and sequence homology data that indicated the monkey was more relevant than rat, the cynomolgus monkey was selected for repeat-dose ocular toxicology studies of FabA.

The repeat dose ocular toxicology studies included ophthalmic examinations (OE), IOP, electroretinogram (ERG), ocular histopathology, and the measurement of FabA in serum and vitreous for TK analyses. Additionally, FabA PD properties were characterized by measurement of C1q in vitreous (for all repeat dose studies) and ocular tissues (in two dose studies), and the inhibition of C1q-dependent hemolysis in serum (in two dose studies).

### Single-Dose Toxicity

IVT administration of FabA was well tolerated in the single-dose (rat and cynomolgus monkey) ocular toxicology studies. In these studies, the NOAELs for rat and cynomolgus monkey were considered to be 0.05 mg/eye (equivalent to 10 mg human dose) and 5 mg/eye (equivalent to 10 mg human dose), respectively, which were the highest doses evaluated in each study and are equivalent (2.5 mg/mL) when corrected for vitreous volume (rat 0.02 mL, monkey 2 mL).

### GLP Single-Dose Ocular Toxicity Study of FabA by Intravitreal Injection in Sprague Dawley Rats

In this single dose GLP rat ocular toxicology study, vehicle or FabA was administered at doses of 0.01 mg/eye (equivalent to 2 mg human dose) and 0.05 mg/eye (equivalent to 10 mg human dose) by IVT injection once bilaterally to young adult male rats. FabA treated animals were terminated at Day 1 (6 hours post dose), Day 3, Day 7, Day 10, Day 20, Day 30 and all vehicle control animals were terminated on Day 30. All animals survived until scheduled necropsy.

Standard safety parameters were included in this study. Blood samples were collected at termination and terminal vitreous samples were obtained for TK analysis. Additionally, ophthalmic examinations (OEs), including IOP, and ocular histopathology were evaluated.

No FabA-related changes were noted in any safety parameter evaluated, including OEs, IOPs, and ocular histopathology.

Vitreous exposure to FabA was confirmed by TK in treated animals for 6 hours (first collection) to 144 hours post dose at both 0.01 mg/eye (equivalent to 2 mg human dose) and 0.05 mg/eye (equivalent to 10 mg human dose). Serum exposure to FabA was confirmed on TK in treated animals (2 to 48 hours post dose only) at 0.01 and 0.05 mg/eye.

No adverse effects considered related to FabA were observed at any dose level, including 0.05 mg/eye, the highest dose evaluated, were observed in this study. Based on these results, the NOAEL was 0.05 mg/eye (2.5 mg/mL in vitreous).

### Non-GLP Single-Dose Ocular Toxicity Study of FabA by Intravitreal Injection in Cynomolgus Monkeys

In this single dose non-GLP cynomolgus monkey ocular toxicology study, vehicle or FabA was administered bilaterally by IVT injection at doses of 1 mg/eye (equivalent to 2 mg human dose) and 5 mg/eye (equivalent to 10 mg human dose) to young adult female cynomolgus monkeys. FabA treated animals were terminated at Day 1 (6 hours post dose), Day 3, Day 7, Day 10, Day 20, Day 30. All vehicle control animals were terminated on Day 30 and all animals survived until scheduled necropsy.

Standard safety parameters including OE, IOP, and ocular histopathology were assessed in this study. Additionally, blood samples were collected throughout the study, and terminal vitreous samples for TK and PD were analyzed.

FabA-related changes were limited to non-adverse findings that were not associated with inflammation. These findings included histiocytic infiltrates in the uvea and mild basophilia in the 1 mg/eye dose group. Findings in the 5 mg/eye dose consisted of histiocytic infiltrates in the uvea, and minimal to mild basophilia.

No FabA-related changes were observed in OEs and IOPs. No adverse effects considered related to FabA at any dose level, including 5 mg/eye (the highest dose evaluated) were observed in this study. Based on these results, the NOAEL was considered to be 5 mg/eye (2.5 mg/mL in vitreous).

Exposure to FabA in the vitreous was confirmed on TK in all treated animals for the duration of the study (through Day 30). Serum exposure to FabA was absent at 1 mg/eye, was low and transient at 5 mg/eye, and did not exceed 6 ng/mL (LLOQ 1.25 ng/mL). C1q was absent in all FabA treated animals in the vitreous through Day 30.

### Repeated-Dose Toxicity Studies

In the repeat-dose ocular toxicity studies, FabA was administered at least once every 4 weeks by IVT injection. Repeat-dose administration of FabA was well tolerated in the cynomolgus monkey. In the initial repeat-dose GLP ocular toxicology studies, the NOAEL for the cynomolgus monkey was 5 mg/eye (equivalent to 10 mg human dose) once monthly for two doses, the highest dose evaluated. In the 26-week chronic ocular toxicology study in cynomolgus monkeys, adverse ocular changes were associated with the double injection procedure and/or determined to be ADA-mediated and not a direct effect of FabA IVT administration, thus the NOAEL was determined to be 2.5 mg/eye (equivalent to 5 mg human dose) biweekly or once monthly in cynomolgus monkeys for 13 or 7 doses, respectively.

### 6-Week GLP Repeated-Dose Ocular Toxicity Study of FabA by Intravitreal Injection in Cynomolgus Monkeys

Standard safety parameters were included in this study, and blood samples were collected throughout the study. Terminal vitreous samples for TK and PD analysis and terminal optic nerve sections for TK and PD analysis were collected as well. Additionally, OEs, IOPs, ERGs, and ocular histopathology were evaluated.

FabA findings determined to not be adverse were limited to one high dose (2.5 mg/eye) (equivalent to 5 mg human dose) female, which had minimal basophilic/blue-staining of the vitreous with no associated inflammation (referred to as basophilia). Importantly, there were no FabA-related changes noted in OEs, IOPs, and ERGs. TK confirmed exposure to FabA in all treated animals in the vitreous for the duration of the study and through recovery (30 days post the last dose).

Serum exposure was not measurable at 1 mg/eye (equivalent to 2 mg human dose), was low and transient (12 to 48 hours post the first dose, 6 to 168 hours post the last dose) at 2.5 mg/eye (equivalent to 5 mg human dose) and did not exceed 8 ng/mL (LLOQ 1.25 ng/mL). PD confirmed the absence of C1q in all treated animals in the vitreous, when FabA levels were ˜100 ng/mL. FabA ADAs were detected in animals at the 1 mg/eye (equivalent to 2 mg human dose) (6 of 12 animals) and 2.5 mg/eye (equivalent to 5 mg human dose) (7 of 12 animals), but there was no clear impact of ADA on FabA exposure in serum or vitreous.

### 6-Week GLP Repeated-Dose Ocular Toxicity Study of FabA by Intravitreal Injection in Cynomolgus Monkeys

In this 6-week GLP cynomolgus monkey ocular toxicology study, vehicle or FabA was administered bilaterally via IVT injection at a dose of 5 mg/eye (equivalent to 10 mg human dose) every four weeks (on Days 1 and 29) to young adult male and female cynomolgus monkeys, followed by a 4-week recovery period. All main study animals were terminated on Day 44 and all recovery animals were terminated on Day 59/60. All main study and recovery animals survived until scheduled necropsy.

Standard safety parameters were included in this study (with the exception of systemic histopathology), and blood samples were collected throughout the study, as well as terminal vitreous samples for TK and PD analysis. ADA and aqueous humor samples were collected and archived. Additionally, OEs, IOPs, ERGs, and ocular histopathology were evaluated.

No FabA-related changes were noted in any safety parameter evaluated including OEs, IOPs, ERGs, and ocular histopathology. Minimal-mild basophilic/blue-staining of the vitreous with no associated inflammation (referred to as basophilia) was observed in both treated and control animals and thus was not considered related to FabA.

TK confirmed exposure to FabA in all treated animals in the vitreous for the duration of the study and through recovery (30 days post the last dose). The absence of FabA was confirmed in the serum and vitreous of control animals. PD confirmed the absence of C1q in all treated main study animals in the vitreous on Day 44. On Day 59, 2/4 recovery animals had measurable C1q in the vitreous. FabA ADAs were detected in animals at the 5 mg/eye (equivalent to 10 mg human dose) (9 of 10 animals) group, but there was no clear impact of ADA on FabA exposure in serum or vitreous.

Inhibition of C1q-dependent hemolysis of >80% was achieved 24-48 hours after FabA administration and returned towards baseline thereafter.

C1q levels were also significantly decreased in the retina, choroid and optic nerve head on Day 44, and continued to be reduced in the retina and choroid, but not the optic nerve head on Day 59.

No adverse effects considered related to FabA at any dose level, including 5 mg/eye (equivalent to 10 mg human dose) (the highest dose evaluated) were observed in this study. Based on these results, NOAEL was 5 mg/eye (equivalent to 10 mg human dose) (2.5 mg/mL in vitreous).

### 26-Week GLP Repeated-Dose Ocular Toxicity Study of FabA by Intravitreal Injection in Cynomolgus Monkeys with 10-Week Recovery

In this 26-week GLP cynomolgus monkey ocular toxicology study, vehicle or FabA was administered bilaterally via IVT injection at a dose of 2.5 mg/eye (equivalent to 5 mg human dose) once monthly, 5 mg/eye (equivalent to 10 mg human dose) once monthly, and 5 mg/eye once every other week (biweekly) to young adult male and female cynomolgus monkeys, followed by a 10-week recovery period. Single injection of 50 μL corresponded to 2.5 mg/eye, or double injections totaling 100 μL (two 50 μL injections separated by 10 min corresponded to 5 mg/eye) every 2 weeks (13 dosing periods) or every 4 weeks (7 dosing periods). All main study animals were terminated on Day 184 and all recovery animals were terminated on Day 242/243. All main study and recovery animals survived until scheduled necropsy.

Dose holidays or cessation of dosing occurred in animals in the control, 5 mg/eye (equivalent to 10 mg human dose) once monthly, and 5 mg/eye biweekly dose groups. Double injections in these groups were discontinued due to adverse findings detected by OEs and were considered related to the procedure and high dose volume. Beginning on Day 71 of the study the 5 mg/eye once monthly group was dosed 2.5 mg/eye (equivalent to 5 mg human dose) once monthly (referred to as 5/2.5 mg/eye once monthly), and 5 mg/eye biweekly was dose 2.5 mg/eye biweekly (referred to as 5/2.5 mg/eye biweekly). Dose holidays continued in these dose groups (including control) after the double injections were discontinued, which were procedural and/or ADA-related as described below. There were no dose holidays in the 2.5 mg/eye once monthly group (low dose group).

Standard safety parameters were included in this study (with the exception of systemic histopathology), and blood samples were collected throughout the study, as well as terminal vitreous samples for TK and PD analysis. ADA and aqueous humor samples were collected and archived. Additionally, OEs, IOPs, ERGs, ocular histopathology, and immunohistochemistry (IHC) for the detection of deposited immune complexes in globes were evaluated.

No FabA-related changes were noted in body weights, food consumption, electroretinography, tonometry, and clinical pathology.

Ocular clinical signs and ophthalmic examination findings considered related to FabA were limited to eyeball opacity (likely due to opacity in the anterior chamber, lens capsule, and/or posterior chamber) and the presence of cells and/or pigment. The presence of these findings in animals that did not have ADA detected in serum (4 of 12 Group 2 animals, 2 of 12 Group 3 animals, and 2 of 12 Group 4 animals) indicates a relationship to FabA. Findings considered related to ADA and potentially to immune complex deposition tended to be more severe and included aqueous flare and the presence of vitreal haze, altered pupillary light reflex, and retinal vessel attenuation.

Following IVT administration of FabA to male and female monkeys systemic exposures as measured by serum FabA concentrations were transient, low, and did not exceed 86.3 ng/mL after dosing on Day 85, or 60.81 ng/mL after the last dose on Day 169. TK confirmed exposure to FabA in nearly all treated animals that received FabA through Day 169, which corresponded with vitreous C1q not being detectable, with the exception of some animals with dose holidays. On Day 242/243 after the 10-week dose-free recovery period FabA vitreous concentrations were not measurable, and C1q concentrations were detectable in all treated dose groups. The absence of FabA was confirmed in the serum and vitreous of control animals.

The presence of anti-FabA antibodies in serum samples was confirmed in 4 of 12 Group 1 (Control) animals, 8 of 12 Group 2 animals, 10 of 12 Group 3 animals, and 10 of 12 Group 4 animals. Two Group 1 animals confirmed positive at a single time point per animal after Day 1, whereas FabA-treated ADA-positive animals were identified at 3 or 4 or more time points (a total of 4 or 5 samples were collected for main study and recovery animals, respectively). There was no clear impact of ADA on FabA exposure in serum or vitreous.

At the terminal euthanasia on Day 184, microscopic changes consistent with an ADA-mediated immune response to FabA were observed in the right eye at 5/2.5 mg/eye monthly and biweekly (mid and high dose groups, respectively). Intraocular changes related to inflammation included mild mixed cell infiltration of the ciliary body and vitreous chamber, minimal to moderate fibrosis (severity proportional to dose frequency) within the vitreous chamber, and minimal to mild posterior lens degeneration (severity proportional to dose frequency). Minimal perivascular mononuclear cell infiltrates were also observed within the posterior retina in one female each at 5/2.5 mg/eye biweekly and monthly treatment groups. Minimal to mild, mononuclear cell infiltration was observed within the periocular limbus of animals administered 5/2.5 mg/eye biweekly and monthly, with severity proportional to dose frequency.

At the recovery euthanasia on Day 242/243, microscopic changes in the right eye related to the ADA-mediated immune response to FabA were limited and minor at 5/2.5 mg/cyc monthly, while additional changes persisted or developed at 5/2.5 mg/cyc biweekly. Minimal histiocytic infiltration of the vitreous chamber and uvea and increased basophilia of the vitreous chamber were exclusive to animals administered FabA 5/2.5 mg/eye biweekly and/or monthly at recovery. These changes were similar to those observed in control animals at the terminal necropsy where they were considered related to minor inflammation/disruption of the anterior vitreous associated with the IVT injection procedure. However, the persistence of these changes after the recovery period, the resolution of these changes in control animals at recovery, and the presence of more severe inflammatory changes at the terminal necropsy in animals administered FabA 5/2.5 mg/eye biweekly and monthly indicated that these changes at recovery more likely represented resolving inflammation related to an ADA-mediated immune response to FabA rather than lingering effects of the injection procedure. Minimal mononuclear cell infiltration of the periocular limbus persisted at both 5/2.5 mg/eye biweekly and monthly.

Minimal mixed cell infiltration and fibrosis of the vitreous chamber persisted at 5/2.5 mg/eye biweekly, while moderate decreased cellularity and hemosiderin pigment of the retina developed. Minimal perivascular mononuclear cell infiltration of the retina at the optic disk was also observed at 5/2.5 mg/eye biweekly. These changes were also considered secondary to an ADA-mediated response to FabA.

Pathologically adverse microscopic changes were considered secondary to ADA-mediated inflammation and included fibrosis within the vitreous chamber, lenticular degeneration, and decreased cellularity of the retina at 5/2.5 mg/eye biweekly and monthly.

There were no microscopic changes related to FabA 2.5 mg/eye monthly at either the terminal or recovery euthanasia.

Immunohistochemistry was conducted for 2 of 12, 4 of 12, and 6 of 12 animals from Groups 1, 3, and 4, respectively. Evaluation revealed the presence of immunohistochemically detected granular deposits containing FabA, monkey IgG, IgM, and/or C3 within the left eye of 4 of 10 treated animals in the mid dose 5/2.5 mg/eye monthly (2 of 4 animals) and high dose 5/2.5 mg/eye biweekly (2 of 6 animals) selected for IHC. These intramural vascular deposits were present in association with perivascular inflammatory cellular infiltrates similar to those observed with hematoxylin and eosin evaluation of the right eye. Other microscopic changes observed within the right eye were consistent with secondary changes associated with this immune response to FabA in the monkey. Although ocular immune complex deposits were not observed in all animals selected for immunohistochemistry, including some with negative serum ADA as well, this was not unexpected as deposit identification may vary with tissue sectioning and serum ADAs are not always present in animals with microscopic evidence consistent with immune complex pathology. Additionally, in some animals with multiple dose holidays, ADA and/or immune complexes may have cleared prior to analyses. The presence of immunohistochemically confirmed deposits even in a subset of animals was considered the most convincing weight of evidence that similar and pathogenetically consistent pathology observed in the right eye was likely related to an immune response to FabA.

In this 26-week chronic ocular toxicology study in cynomolgus monkeys, adverse ocular changes were determined to be related to the double injection procedure and/or ADA-mediated and not a direct effect of FabA IVT administration, thus the NOAEL was determined to be 2.5 mg/eye (equivalent to 5 mg human dose) biweekly or once monthly in cynomolgus monkeys for 13 or 7 doses, respectively.

### Example 2

### Evaluating FabA in Clinical Studies

FabA Drug Product is a sterile, isotonic liquid for IVT injection. A Phase 1 first-in-human, open-label, dose-escalation study (FabA-GLA-01) was conducted to evaluate the initial safety and tolerability of a single IVT injection of FabA in patients with primary open-angle glaucoma.

A Phase 1b, randomized, double-masked study (FabA-GLA-02) was conducted to evaluate the safety and tolerability of repeat IVT injections of FabA in patients with primary open-angle glaucoma.

Results of both studies found that single (1 to 5 mg/eye) (equivalent to 2-10 mg human dose) and repeat doses (2.5 and 5 mg/eye, 2 doses separated by 4 weeks) of FabA IVT were well tolerated in glaucoma patients; no serious or significant adverse events (AEs) were reported. Ocular AEs in patients treated with FabA in these studies included conjunctival hyperemia, conjunctival hemorrhage, and eye irritation and only occurred in the treated eye. In the Phase 1b study, ocular AEs in patients in the Sham group included eye pain, foreign body sensation in eyes, ocular hyperemia, and vision blurred. No systemic AEs occurred that were considered related to FabA IVT treatment.

Single IVT injections of 2.5 mg (equivalent to 5 mg human dose) and 5 mg (equivalent to 10 mg human dose) FabA inhibited free C1q for at least 29 days in aqueous humor (Study FabA-GLA-02).

### Pharmacokinetics and Pharmacodynamics in Humans

### Ocular Pharmacokinetics and Pharmacodynamics

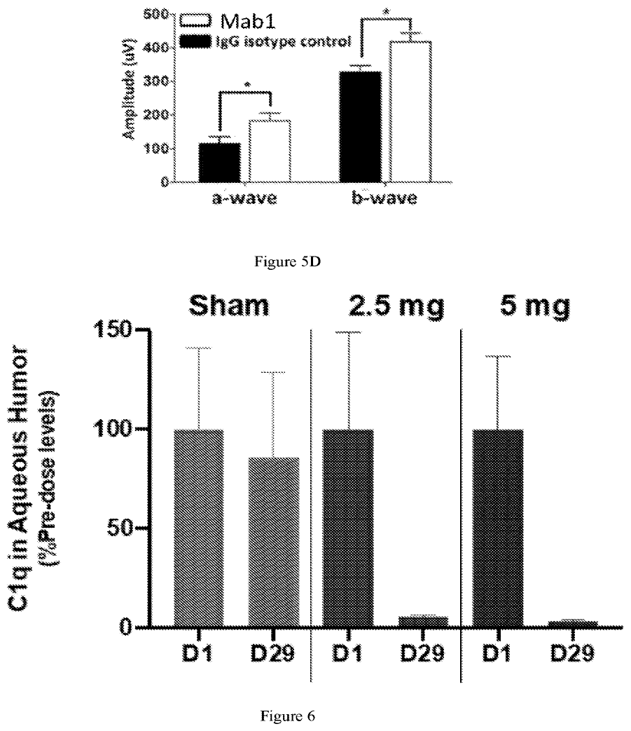

FabA-GLA-02 is a Phase 1b study in which aqueous humor was sampled to assess PK and PD. Subjects were administered two IVT injections of sham, 2.5 mg/eye FabA (equivalent to 5 mg human dose), or 5 mg/eye (equivalent to 10 mg human dose) FabA separated by 29 days. In this study, aqueous humor was sampled predose and 29 days following the first FabA dose, prior to the second dose. Free FabA was detected in the aqueous humor of all treated patients on Day 29 (D29). In parallel, both dose levels of 2.5 mg/eye and 5 mg/eye FabA inhibited free C1q for at least 29 days in aqueous humor (FIG. 6).

### Systemic Pharmacokinetics and Pharmacodynamics

FabA-GLA-01 is a single dose Phase 1 study in which serum FabA and C1q were sampled predose and at 3 hours postdose. FabA-GLA-02 is a multidose Phase 1b study in which serum and FabA and C1q were sampled predose and at 3 hours postdose for each of 2 doses separated by 29 days. FabA was generally not detectable in systemic circulation after single or repeat IVT injections at any dose level studied in either the Phase 1 or Phase 1b clinical studies. Similarly, no changes in circulating free C1q were detected in either study.

As described below, the 5 mg/eye (equivalent to 10 mg human dose) dose level was well tolerated as single or two doses separated by 29 days in FabA clinical studies. As described above, in the Phase 1b study, single doses of FabA at 2.5 mg (equivalent to 5 mg human dose) and 5 mg (equivalent to 10 mg human dose) inhibited free C1q in aqueous humor for at least 29 days (FIG. 6).

### Safety and Efficacy

### Phase 1 Dose Escalation (FabA-GLA-01)

This was a Phase 1, open-label, dose-escalation study evaluating the safety/tolerability and PK of a single IVT injection of FabA in patients with primary open-angle glaucoma. Eligible patients were adults with mean deviation of 3 to 18 dB on a reliable visual field test who were able to perform a reliable visual field test in the study eye with a cutoff of 33% for fixation losses and 33% for false-positive response rates using the Humphrey Field Analyzer-Swedish Interactive Threshold Algorithm (HFA-SITA) 24-2 fast algorithm, and had IOP <21 mmHg on a stable IOP treatment regimen in the study eye for >4 weeks prior to dosing. Nine patients were assigned to 3 cohorts, with 3 patients enrolled per cohort as follows:
Cohort 1=1.0 mg/eye, single dose (0.02 mL)×1 dose
Cohort 2=2.5 mg/eye, single dose (0.05 mL)×1 dose
Cohort 3=5.0 mg/eye, single dose (0.10 mL)×1 dose

After screening, 3 eligible patients were enrolled into the lowest open cohort, with enrollment in the next cohort initiated only after tolerability and short-term safety had been demonstrated at the preceding lower dose. All patients within each cohort were required to complete a minimum of a 15-day, safety observation period before patients in the next cohort could be injected. No dose-limiting toxicities (DLTs) were reported during the study.

Nine patients were enrolled, treated, and completed the study.

### Safety

Ocular treatment-emergent adverse event (TEAEs) included conjunctival hyperemia (all dose levels), conjunctival hemorrhage (2.5 mg/eye only), and eye irritation (1 mg/eye only) and only occurred in the study eye.

The only systemic TEAE experienced in the study was sinusitis.
All TEAEs were mild in severity.
There were no serious or significant TEAEs.
No patients discontinued treatment or withdrew from the study due to a TEAE.
IOP returned to normal (within 5 mmHg of immediate pre-injection IOP or <21 mm Hg) within 30 minutes in 9 of 9 patients.
No patient showed any evidence of anti-FabA antibodies.

### Overall Summary/Conclusions

In this study in patients with stable glaucoma, single IVT doses of FabA were well tolerated up to 5 mg/eye. The ocular AEs reported were similar to those reported with IVT administration of approved drugs. No safety signals with FabA were observed.

FabA was generally not detectable in the systemic circulation and no changes in circulating free C1q were detected after single IVT administration.

### Phase 1b (FabA-GLA-02)

This was a double-masked, randomized, sham-controlled study to evaluate two dose levels of FabA vs. sham injection, administered as repeat IVT injections in patients with primary open-angle glaucoma. Eligible patients were adults with a mean deviation of −3 to −24 dB on a reliable visual field test in the study eye who were able to perform a reliable visual field test in the study eye with a cutoff of 33% for fixation losses and 33% for false-positive response rates using the HFA-SITA fast algorithm, had IOP <21 mmHg at screening and on Day 1, and were on a stable IOP treatment regimen for ≥4 weeks prior to injection, with no anticipated change in IOP treatment regimen during the study. Patients received two injections, 4 weeks apart, and were followed for a total of 12 weeks for evaluation of the safety, tolerability, PK, PD, immunogenicity, and ongoing exploratory evaluations. Patients were randomly assigned (1:1:1) to one of 3 cohorts (5 patients per cohort were planned), as follows:
Dose Level 1=2.5 mg/eye, single dose (0.05 mL)×2 doses
Dose level 2=5.0 mg/eye, single dose (0.10 mL)×2 doses
Sham=0 mg/eye×2 doses

Eighteen patients were randomized (7 to the 2.5 FabA group, 5 to the 5.0 mg FabA group, and 6 to the sham group) and 17 patients were treated. One patient in the 2.5 mg dose group was randomized, but was not treated. Sixteen patients completed the study.

### Safety

Ocular TEAEs experienced by patients treated with FabA included conjunctival hyperemia (2.5 and 5 mg/eye), conjunctival hemorrhage (5 mg/eye only), and eye irritation (5 mg/eye only); none of these TEAEs were experienced by patients in the Sham group. Ocular TEAEs in the Sham group included eye pain, foreign body sensation in eyes, ocular hyperemia, and vision blurred and occurred in 1 patient each.

Systemic TEAEs were experienced in the study; none were considered related to study treatment by the investigator.
All TEAEs were mild in severity.
All but one of the TEAEs reported occurred after the first dose but prior to the second administration of study treatment.
There were no serious or significant TEAEs.
No patient discontinued treatment or withdrew from the study due to a TEAE.
IOP returned to normal (<21 mm Hg) for 16/17 patients within 30 minutes of the IVT injection and within 45 minutes for the remaining patient.
Of the 11 patients dosed intravitreally with FabA, 6 patients tested positive during at least one time point. One patient was ADA positive with modest increase in titer over time, and the remaining 5 patients were positive at all time points, including predose, without any change in titer over time. One Sham patient was positive for ADA at all time points, including predose, without any change in titer over time. Together, these data suggest an unclear relationship between ADA measurement and FabA dosing.

### Overall Summary/Conclusions

In patients with stable glaucoma, 2 IVT doses of FabA separated by 4 weeks were well tolerated up to 5 mg/eye. The ocular AEs reported were similar to those reported with IVT administration of approved drugs. In this study, no safety signals with FabA were observed.

Single doses of FabA IVT (2.5 and 5 mg/eye) inhibited free C1q in aqueous humor for at least 29 days.

### Example 3

### A Phase 2, Multicenter, Randomized, Parallel-Group, Double-Masked, 4-Arm, Sham-Controlled Study of the Efficacy, Safety, and Tolerability of FabA Administered by Intravitreal Injection in Patients with Geographic Atrophy (GA) Secondary to Age-Related Macular Degeneration (AMD)

### Rationale

### Brief Summary

This study is being conducted in patients with GA secondary to AMD. The purpose of the study is to determine if intravitreal (IVT) injections of FabA once every month (EM) or once every other month (EOM) for 12 months reduces GA lesion growth rate. The study consists of a 30-day screening period and a 12-month treatment period, followed by a 6-month (off treatment) follow-up period. The total duration of patient participation is 19 months. Patients visit the clinic each month during the 12-month treatment period for treatment and/or safety assessments.

Approximately 240 patients are enrolled and randomly assigned to one of 4 treatment arms so that approximately 204 patients are evaluable at Month 12 for the primary analysis (primary analysis is based on modified intent-to-treat [ITT]).

### Intervention Groups and Duration

Study intervention assignment is based on randomization (2:2:1:1). Patients are assigned to one of the following treatment arms. Dose level is fixed and are not modified.
Arm 1=FabA 5.0 mg/eye (0.10 mL) once every month (EM) for 12 months (12 doses)
Arm 2=FabA 5.0 mg/eye (0.10 mL) once every other month (EOM) for 12 months (6 doses)
Arm 3=Sham injection EM for 12 months (12 Sham injections)
Arm 4=Sham injection EOM for 12 months (6 Sham injections)

### Injection

FabA/Sham administration is completed by the injecting physician using aseptic technique.

All patients randomized to FabA receive 5.0 mg/eye IVT (at a fixed volume of 0.10 mL) once every month or once every other month for 12 months.

### Post-Injection

Immediately following drug administration, the injecting physician assesses hand motion vision or central retinal artery perfusion visualization. If necessary, rule out other causes of vision loss such as vitreous hemorrhage. If needed, perform digital massage, and administer topical/oral IOP lowering medications, until hand motion vision or central retinal artery perfusion is observed.

IOP (tonometry) is evaluated in the study eye only, 30 minutes after drug administration and, if elevated, every 15 minutes thereafter until IOP <25 mmHg.

### Pharmacokinetics, Pharmacodynamics, and Immunogenicity

Blood samples for PK (FabA serum concentrations) and PD evaluations (serum C1q concentrations and plasma concentrations of other biomarkers) are collected within 30 minutes before dosing and 3 hours (±15 min) after dosing at the visits.

Samples for immunogenicity testing (ADA) are collected pre-injection during the site visits. Additionally, a sample for ADA is collected at Week 2 at the site or a home health visit.

Pharmacokinetics: This test requires scrum. Blood samples arc collected for measurement of serum concentrations of FabA.

Pharmacodynamics: This test requires serum and plasma. Serum concentrations of C1q and plasma concentrations of exploratory complement biomarkers are analyzed.

Immunogenicity: This test requires serum. Immunogenicity is assessed by analysis of serum anti-drug (FabA) antibodies (ADA).

### Procedure for Intravitreal Injection

### Preparation of FabA

Withdraw the entire volume of FabA (approximately 0.3 mL) from the sterile vial of FabA, using a sterile 1.0 cc syringe with a 19-gauge×1½ inch, 5 micron, filter needle.

Replace the filter needle with a 30-gauge×½ inch injection needle. Expel the excess volume of FabA from the syringe just prior to injection, leaving only the required injection volume in the syringe.

The dose volume of FabA is fixed at 0.10 mL once every month (EM) for 12 months (12 doses) or once every other month (EOM) for 12 months (6 doses).

### Preparation for Intravitreal Injection

1. Verify the study eye.

2. Measure and record the pre-op intraocular pressure (IOP) in the study eye before injection. Perform tonometry on study eye only. The IOP must be ≤21 mmHg to proceed. If >21 mmHg, reschedule the injection of FabA and manage the IOP at the Investigator's discretion.

3. Apply 1 drop of topical phenylephrine hydrochloride ophthalmic solution 2.5% to the study eye 30 minutes before the injection to allow visualization of the posterior pole after the injection, if necessary.

4. Immediately before injection:
Have the patient lie back in the examination chair with the neck well supported.

### Intravitreal Injection

1. Hand-washing, sterile gloves, and surgical mask are required for the injection.

2. Apply topical proparacaine 0.5% to the study eye.

3. Apply povidone-iodine 10% to the eyelashes and eyelid margins. Avoid extensive massage of the eyelids either pre- or post-injection to avoid meibomian gland expression.

4. Retract the eyelids away from the intended injection site for the duration of the procedure. Use of a speculum is recommended.

5. Apply povidone-iodine 5% to the conjunctival surface, including the intended injection site.

6. For injection with FabA: Insert the needle perpendicular to the sclera, 3.5 to 4 mm posterior to the limbus, between the vertical and horizontal rectus muscles. Apply a sterile cotton-tip applicator over the injection site immediately following removal of the needle to reduce vitreous reflux.

7. Sham injection: The preparation and post-injection care for the sham injection is identical to injection with FabA. The sham injection is performed by applying pressure to the eye at the location of a typical intravitreal injection using the blunt end of an empty syringe without a needle.

### Post-Intravitreal Injection

1. Patients are to remain in the clinic after injection for ocular evaluations and safety follow-up.

2. Assess hand motion vision or central retinal artery perfusion immediately and rule out other causes of vision loss such as vitreous hemorrhage. Perform digital massage and administer topical/oral IOP lowering medications until hand motion vision or central retinal artery perfusion is observed, if no other cause is found.

3. Obtain IOP measurement in the study eye only at 30 minutes post-injection and, if elevated, every 15 minutes until IOP <25 mmHg. Intraocular pressure in excess of 30 mmHg for more than 15 minutes should be treated at the physician's discretion.

4. Topical antibiotics are not required.

### INCORPORATION BY REFERENCE

Each of the patents, published patent applications, and non-patent references cited herein are hereby incorporated by reference in their entirety.

### Equivalents

Those skilled in the art will recognize, or be able to ascertain using no more than routine experimentation, many equivalents to the specific embodiments of the invention described herein. Such equivalents are intended to be encompassed by the following claims.

1. A method of treating an ocular disease in a human patient, comprising administering to the patient a composition comprising about 1 mg to about 10 mg of an anti-C1q antibody via an intravitreal injection,
wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7; and a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

2. The method of claim 1, wherein the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7.

3. The method of claim 2, wherein the light chain variable domain comprising an amino acid sequence selected from SEQ ID NO: 4 and 35-38.

4. The method of claim 1, wherein the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11.

5. The method of claim 4, wherein the heavy chain variable domain comprising an amino acid sequence selected from SEQ ID NO: 8 and 31-34.

6. The method of claim 1, wherein the antibody is a monoclonal antibody, a humanized antibody, a human antibody, a chimeric antibody, an antibody fragment, or antibody derivative thereof.

7. The method of claim 6, wherein the antibody is an antibody fragment and the antibody fragment is a Fab fragment, a Fab′ fragment, a F(ab′)2 fragment, a Fv fragment, a diabody, or a single chain antibody molecule.

8. The method of claim 7, wherein the Fab fragment comprises a heavy chain Fab fragment of SEQ ID NO: 39 and a light chain Fab fragment of SEQ ID NO: 40.

9. The method of claim 1, wherein the antibody is administered once a week.

10. The method of claim 1, wherein the antibody is administered once every other week.

11. The method of claim 1, wherein the antibody is administered once every three weeks.

12. The method of claim 1, wherein the antibody is administered once a month.

13. The method of claim 1, wherein the antibody is administered once every four weeks.

14. The method of claim 1, wherein the antibody is administered once every six weeks.

15. The method of claim 1, wherein the antibody is administered once every 8 weeks.

16. The method of claim 1, wherein the antibody is administered once every other month.

17-20. (canceled)

21. The method of claim 9, wherein the antibody is administered for at least 3 months, at least 4 months, at least 6 months, at least 7 months, at least 8 months, at least 9 months, at least 10 months, at least 11 months, or at least 12 months.

22. (canceled)

23. The method of claim 1, wherein the administered composition comprises about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg of the anti-C1q antibody.

24-30. (canceled)

31. The method of claim 1, wherein the ocular disease is glaucoma or age-related macular degeneration.

32. The method of claim 31, wherein the age-related macular degeneration is geographic atrophy.
